(19) United States
(12) Patent Application Publication
(10) Pub. No.: US 2026/0321365 A1
(43) Pub. Date: Oct. 15, 2026

(54) METHODS AND SYSTEMS FOR AI ENHANCED CLINICAL TRIAL PROCESSES

(71) Applicant: SKR Carolina LLC, Cary, NC (US)

(72) Inventor: Maithraeyi Annamalai, Cary, NC (US)

(21) Appl. No.: 19/468,115

(22) Filed: Feb. 3, 2026

Related U.S. Application Data
(60) Provisional application No. 63/752,974, filed on Feb. 3, 2025.

(51) Int. Cl. G16H 10/20; G16H 50/70

(52) U.S. Cl. CPC G16H 10/20; G16H 50/70

(57) ABSTRACT

A computer-implemented method includes determining, using a first Artificial Intelligence (AI) model that is trained using multi-omic features associated with a clinical condition, molecular markers that distinguish between a patient affected with the clinical condition and a healthy individual; clustering patients based on the molecular markers to determine which of the molecular markers drive the formation of different cluster groups; prioritizing the molecular markers that drive the formation of different cluster groups based on biological relevance and clinical applicability; and determining, using a second AI model that is trained based on historical prioritized molecular markers and labeled drug response and disease state classifications, one or more patients that respond to a drug for the clinical condition, one or more patients that do not respond to the drug for the clinical condition, or a disease state classification for each of one or more patients.

### CROSS-REFERENCE TO RELATED APPLICATION

The present application claims priority to U.S. Provisional Patent Application Serial No. 63/752,974, filed Feb. 3, 2025, the entire content of which is incorporated by reference herein as if set forth in its entirety.

### FIELD

The present disclosure relates to systems and methods for enhancing clinical trial processes.

### BACKGROUND

The statements in this section merely provide background information related to the present disclosure and may not constitute prior art.

Current approaches to drug development and clinical trials rely on retrospective analysis, which may limit the ability to dynamically adjust trials in real time. Platforms such as QIAGEN IPA, Metacore, and GeneSpring focus primarily on post-hoc research-based analysis and may fail to provide real-time integration of multi-domain data. Additionally, existing platforms may not adapt to non-traditional therapeutic areas, such as nutraceuticals, gene-editing therapies, and real-world evidence studies that fall outside the scope of traditional clinical trials.

### SUMMARY

According to some embodiments of the disclosure, a computer-implemented method comprises performing, by one or more processors, operations comprising: determining, using a first Artificial Intelligence (AI) model that is trained using multi-omic features associated with a clinical condition, molecular markers that distinguish between a patient affected with the clinical condition and a healthy individual; clustering patients based on the molecular markers to determine which of the molecular markers drive the formation of different cluster groups; prioritizing the molecular markers that drive the formation of different cluster groups based on biological relevance and clinical applicability; and determining, using a second AI model that is trained based on historical prioritized molecular markers and labeled drug response and disease state classifications, one or more patients that respond to a drug for the clinical condition, one or more patients that do not respond to the drug for the clinical condition, or a disease state classification for each of one or more patients.

In other embodiments, the multi-omic features comprise: epigenomic data, genomic data, transcriptomic data, or proteomic data.

In still other embodiments, the method further comprises: processing the multi-omic features to impute estimates of missing data and to remove low-information features.

In still other embodiments, the method further comprises: processing the multi-omic features to remove demographic effects.

In still other embodiments, the method further comprises: reducing the dimensionality of the multi-omic features by removing a subset of the multi-omic features based on a Principal Component Analysis (PCA).

In still other embodiments, the first AI model is configured to place greater importance on ones of the molecular markers that are more informative than other ones of the molecular markers.

In still other embodiments, determining the molecular markers that drive the formation of the different cluster groups comprises: determining the relative of importance of the molecular markers among different cluster groups; and determining a correlation between the molecular markers and clinical attributes.

In still other embodiments, prioritizing the molecular markers comprises: correlating the molecular markers with the severity and progression of the clinical condition; corelating the molecular markers with response to drug treatment; and assessing the involvement of the molecular markers in neuronal pathways and tissue-specific expression.

In still other embodiments, the method further comprises storing one or more persistent storage objects in a computer readable medium, the persistent storage objects comprising: a patient stratification object based on a multi-omic data stream, a clinical data stream, or a non-clinical data stream; a cohort enrichment object based on one or more computational inference models; a dosing schema object; a policy object directed to clinical trial arm allocation, stopping rules, or recruitment targets; or a post clinical trial analysis object comprising reusable decision policies and optimization policies.

In still other embodiments, the method further comprises: storing one or more persistent optimization objects in a computer readable medium, the optimization objects comprising: retrospective learning objects; pre-enrollment learning objects; and interim learning objects.

In still other embodiments, the persistent optimization objects are supplemented based on user input.

In still other embodiments, the persistent optimization objects further comprise justification objects that link optimization objects to biological mechanisms and feature important templates.

In still other embodiments, the persistent optimization objects are generated using one or more data modalities comprising proteomics, genomics, metabolomics, transcriptomics, or longitudinal patient trajectories.

In still other embodiments, the persistent optimization objects further comprise: causal inference models and counterfactual simulations objects based on alternative stratification strategies, enrichment thresholds, and drug dose allocations.

In some embodiments of the disclosure, a system comprises one or more processors; and one or more memories configured to store processor-executable instructions that, when executed by the one or more processors, cause the one or more processors to perform operations comprising: determining, using a first Artificial Intelligence (AI) model that is trained using multi-omic features associated with a clinical condition, molecular markers that distinguish between a patient affected with the clinical condition and a healthy individual; clustering patients based on the molecular markers to determine which of the molecular markers drive the formation of different cluster groups; prioritizing the molecular markers that drive the formation of different cluster groups based on biological relevance and clinical applicability; and determining, using a second AI model that is trained based on historical prioritized molecular markers and labeled drug response and disease state classifications, one or more patients that respond to a drug for the clinical condition, one or more patients that do not respond to the drug for the clinical condition, or a disease state classification for each of one or more patients.

In still further embodiments, the multi-omic features comprise: epigenomic data, genomic data, transcriptomic data, or proteomic data.

In still further embodiments, determining the molecular markers that drive the formation of the different cluster groups comprises: determining the relative of importance of the molecular markers among different cluster groups; and determining a correlation between the molecular markers and clinical attributes.

In still further embodiments, prioritizing the molecular markers comprises: correlating the molecular markers with the severity and progression of the clinical condition; corelating the molecular markers with response to drug treatment; and assessing the involvement of the molecular markers in neuronal pathways and tissue-specific expression.

In still further embodiments, the operations further comprise storing one or more persistent storage objects in a computer readable medium, the persistent storage objects comprising: a patient stratification object based on a multi-omic data stream, a clinical data stream, or a non-clinical data stream; a cohort enrichment object based on one or more computational inference models; a dosing schema object; a policy object directed to clinical trial arm allocation, stopping rules, or recruitment targets; or a post clinical trial analysis object comprising reusable decision policies and optimization policies.

In still further embodiments, the operations further comprise storing one or more persistent optimization objects in a computer readable medium, the optimization objects comprising: retrospective learning objects; pre-enrollment learning objects; and interim learning objects.

It is noted that aspects described with respect to one embodiment may be incorporated in different embodiments although not specifically described relative thereto. That is, all embodiments and/or features of any embodiments can be combined in any way and/or combination. Moreover, other methods, systems, articles of manufacture, and/or computer program products according to embodiments of the disclosure will be or become apparent to one with skill in the art upon review of the following drawings and detailed description. It is intended that all such additional systems, methods, articles of manufacture, and/or computer program products be included within this description, be within the scope of the present inventive subject matter and be protected by the accompanying claims.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram illustrating a first AI system for molecular marker feature generation based on multi-omic and clinical data according to some embodiments of the disclosure;

FIG. 2 is a block diagram illustrating a second AI system for molecular marker processing, feature prioritization, and/or patient stratification based on drug response and/or disease state classification according to some embodiments of the disclosure;

FIG. 3 is a flowchart that illustrates operations for molecular marker feature generation, feature prioritization, and/or patient stratification based on drug response and/or disease state classification in a clinical trial according to some embodiments of the disclosure;

FIG. 4 is block diagram of a data processing system that can be used to implement a system for molecular marker feature generation, feature prioritization, and/or patient stratification based on drug response and/or disease state classification in a clinical trial according to some embodiments of the disclosure;

FIG. 5 is a block diagram of a predictive data analysis computing entity of FIG. 4 according to some embodiments of the disclosure; and

FIG. 6 is a block diagram of an external computing entity of FIG. 4 according to some embodiments of the disclosure.

### DETAILED DESCRIPTION

In the following detailed description, numerous specific details are set forth to provide a thorough understanding of embodiments of the disclosure. However, it will be understood by those skilled in the art that embodiments of the disclosure may be practiced without these specific details. In some instances, well-known methods, procedures, components, and circuits have not been described in detail so as not to obscure the disclosure. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

Embodiments of the disclosure are described herein in the context of a clinical trial for the clinical condition of Amyotrophic Lateral Sclerosis (ALS). While the example embodiments are described herein with respect to ALS, it will be understood that embodiments of the disclosure are not limited to ALS, but are applicable to other clinical conditions of diseases where molecular profiling data is available for patient and control groups.

Embodiments of the disclosure are described herein in the context of processing multi-omic and clinical data. The AI models disclosed herein may be embodied in a variety of different ways including, but not limited to, one or more of the following AI systems: a multi-layer neural network, a machine learning system, a deep learning system, a large language model, a natural language processing system, and/or computer vision system. Moreover, it will be understood that the multi-layer neural network is a multi-layer artificial neural network comprising artificial neurons or nodes and does not include a biological neural network comprising real biological neurons. The AI models described herein may be configured to transform a memory of a computer system to include one or more data structures, such as, but not limited to, arrays, extensible arrays, linked lists, binary trees, balanced trees, heaps, stacks, and/or queues. These data structures can be configured or modified through the processing of monocular image information, text information, and/or music information to improve the efficiency of a computer system when the computer system operates in an inference mode to make an inference, prediction, classification, suggestion, or the like with respect to molecular marker feature generation, feature prioritization, and/or patient stratification based on drug response and/or disease state classification.

The AI system described herein provides a technical improvement over conventional systems due to its biostatistics, bioinformatics, and machine learning capabilities, offering a holistic understanding of disease mechanisms for personalized treatment. Approximately 90% of clinical trials fail, primarily due to lack of efficacy (57%) or adverse reactions (17%), often because molecular differences are not considered. The AI system investigates responders in any phase of a clinical trial, identifying multi-omic biomarkers and molecular pathways associated with drug efficacy and adverse reactions. This data enables users to develop more effective and safer drugs, tailor treatments to patient subgroups, and reduce development costs by focusing on candidates with higher success rates.

Furthermore, about 80% of trials fail to meet their enrollment targets, leading to potential daily revenue losses of up to $8 million for pharmaceutical companies. To mitigate this, the AI system according to embodiments of the disclosure uses data from non-responders in any phase trials to identify alternative pathways that could be targeted by other drugs in the pipeline. This strategy not only creates opportunities for repurposing existing drugs or developing new ones tailored to specific non-responder groups, but also expands market potential and addresses unmet medical needs. Additionally, the AI system according to some embodiments of the disclosure supports regulatory submissions by providing robust data on drug mechanisms and patient selection criteria, thereby accelerating approval processes. This knowledge informs the development of companion diagnostics. The AI system is configured to support the unique workflows of a wet lab, seamlessly integrating and expanding the analysis to include epigenomic, transcriptomic, metabolomic, and lipidomic data tailored to specific diseases.

In some embodiments of the disclosure, the AI system integrates and analyzes multi-omic data, which is complex and voluminous. Conventional systems struggle with the high dimensionality and heterogeneity of multi-omic datasets, but the AI system according to some embodiments of the disclosure leverages advanced biostatistics, bioinformatics, and machine learning techniques to overcome these hurdles. The AI system uses a hypothesis-free methodology to uncover novel biomarkers and molecular pathways without preconceived notions, requiring sophisticated algorithms capable of extracting meaningful patterns from noise, ensuring data integrity, and validating findings across diverse datasets. Molecular characterization of participants to investigational drugs is challenging due to variability in individual responses, necessitating robust computational models that can provide reliable insights. The AI system bridges the gap between vast biological data and actionable clinical insights, pushing the boundaries of precision medicine.

Embodiments of the present disclosure provide an AI system configured to perform patient stratification, dosing, and/or clinical trial optimization routines. The AI system enables deep exploration of multi-omic profiles and biomarker identification from clinical trial participants. The AI system leverages multi-omic data to make real-time adjustments during any phase of clinical trials, unlike QIAGEN IPA, Metacore (Clarivate Analytics), and GeneSpring (Agilent Technologies). While conventional systems use post-hoc, research-based analysis, the AI system described herein integrates real-world, high-throughput biological data, and clinical trial data, thereby allowing trials to be dynamically adjusted as new data emerges, which enhances patient stratification and improves treatment outcomes during ongoing trials.

In some embodiments, the AI system provides real-time clinical trial optimization and patient stratification and identifies interim efficacy signals while trials are ongoing. The AI system integrates complex, high-dimensional datasets with clinical trial data in real time, ensuring the regulatory standards are met while scaling across diverse genetic diseases and other types of diseases. That is, the AI system integrates historical and current patient trial data, and the AI system validates core algorithms, ensuring accuracy and scalability, and improving or optimizing trial design and patient selection. The AI system adjusts trials in real-time by monitoring patient responses, while post-trial analysis offers insights for unsuccessful trials. Additionally, historical analysis identifies biomarkers from previous trials to aid in selecting biomarkers for new trials. In some embodiments, the AI system integrates API-driven data visualization tools that can handle dynamic data updates and provide intuitive insights while balancing real-time performance with data complexity.

In some embodiments, the AI system implements a scalable, adaptable machine learning model that ensures robustness and large-scale application in precision medicine. The AI system uses various deep learning models, such as deep learning and/or neural networks, to capture complex nonlinear relationships in multi-omic data. The AI system incorporates explainable AI (XAI) and/or natural language processing (NLP) techniques to provide transparency in predictions and is trained to ensure model interpretability for clinical decision making, to avoid overfitting, and to reduce computational resource requirements (e.g., quantization levels for various features may be selected in a manner that does not reduce model accuracy below a threshold while reducing processor use and/or memory consumption by other thresholds). That is, one or more of the features may be selected to have different quantization levels, e.g., 64-bit, 32-bit, etc. to reduce the size and complexity of the machine-learned model. Features may be selected for quantization level based on their priority, which can be based on their impact on a classification or prediction of one or more of the AI models. For example, one or more loss functions may be applied based on the different outputs of an AI model relative to various combinations of input features to determine which features have relatively greater importance in generating an accurate output, such as a prediction, estimation, categorization, inference, or the like

In some embodiments, the AI system uses supervised learning, unsupervised learning, reinforcement learning, transfer learning, meta-learning, federated learning, and few-shot learning. As an example, supervised classification (e.g., boosted trees) and/or unsupervised clustering (e.g., k-means clustering) methods may be used for patient stratification and dose-response modeling.

In some embodiments, the AI system stratifies patients of a trial (e.g., ALS (Amyotrophic Lateral Sclerosis) patients) using genomic variants (e.g., VUS, LP, P), corresponding protein metrics, NFL levels (CSF/blood biomarker), ALSFRS-R scores, and other biomarkers. In some embodiments, the AI system leverages proteogenomic biomarker profiles, which offer an understanding of the genetic blueprint's translation into functional proteins and thus provide precision medicine and customizing patient care, to forecast clinical trial outcomes. The AI system utilizes a multi-tier architecture integrating high-throughput sequencing data, advanced bioinformatics pipelines, and machine learning, and the AI system uses modular design principles to ensure scalability and flexibility to thereby enable precise disease characterization, drug response prediction, and therapeutic target identification.

In some embodiments, the AI system may be trained based on various chemical data, biological data, pharmacological data, environmental data, social data, behavioral data, electronic health record (EHR) data, image data, and/or wearable device data. Example training data may include, but is not limited to, phenotypic, genomic, epigenomic, metabolomic, proteomic, transcriptomic, lipidomic metabolomic, digital biomarkers, environmental exposure, single-cell, molecular, cellular, tissue, organ, and/or population data. In some embodiments, the AI system may be continuously trained to further enhance the accuracy and reduce the used computational resources of the AI system.

In some embodiments, the AI system may be trained based on a publicly available dataset, such as AnswerALS, which includes data of 1200+ participants, including 200 controls, and includes longitudinal clinical, genomic, transcriptome, and protein expression data for each participant. Given the large size of various public datasets (e.g., AnswerALS comprises 110 trillion data points), to reduce the computational requirements for feature selection, dimensionality reduction techniques (e.g., PCA, t-SNE) are performed as part of data pre-processing. The AI system uses dimensionality reduction techniques to simplify data, conduct pathway analysis to identify significant biological pathways, and use statistical and meta-analytic methods to integrate data from multiple studies. Additionally, the AI system analyzes treatment response changes over time through longitudinal analysis. The dimensionality reduction techniques, statistical and meta-analytic methods, and/or the longitudinal analysis enables the AI system to analyze biological data, identifying meaningful patterns, relationships, and clinical outcome predictors with high precision and efficiency (e.g., reduced computational resources, such as processor-based and/or memory-based resources). In some embodiments, the AI system applies standard feature engineering techniques to select a small subset of the most statistically significant features for patient stratification.

In some embodiments, once the AI system is trained, the performance may be evaluated using various metrics, such as precision, recall, F1 score, ROC AUC score, Cohen Kappa score, Matthew’s correlation coefficient, and log loss. The parameters (e.g., hyperparameters) of the AI system may be selectively modified until the parameters satisfy a predetermined criterion. In addition to the algorithmic validation using above metrics, the validity may be assessed by running the algorithm against a target cohort (e.g., a database comprising clinical trial data, proprietary data, and/or data associated with published papers), and various parameters of the AI system may be selectively modified until the validity satisfies a predetermined criterion.

While the present disclosure describes an AI system configured to perform patient stratification, dosing, and/or clinical trial optimization routines, it should be understood that similar techniques may be performed to optimize decision making processes in other technical disciplines, such as drug development, non-human clinical trials, real-world evidence studies, nutraceutical applications, wellness treatments, veterinary studies, and gene-editing therapies.

### Example Embodiments

### Patient Stratification and Dosing Using AnswerALS Database

In some embodiments, the AI system uses data from past ALS patients to improve ongoing clinical trials by actively optimizing treatment strategies, focusing on patient stratification, and the AI system is trained based on the data of the AnswerALS database. In some embodiments, the AI system applies data of the AnswerALS database to stratify patients in live trials and/or determine optimal dosing for patients of a different database, where drugs differ across trials. In some embodiments, the AI system creates patient-level (endotype) clustering based on multi-omic molecular signatures, identifies responders and non-responders, and identifies features that are relevant to individual patient clusters.

### Data Sources and Preparation

The training data may include at least one of genomic data (e.g., relevant ALS-related mutations, such as SOD1, C9orf72, TDP 43 and others; proteomic/biomarker data (e.g., NFL levels and other disease markers); other types of multi-omic data; ALSFRS-R scores/clinical data (e.g., data indicating the progression of disease in patients); demographic data (e.g., age, gender, disease onset type, and other relevant characteristics); drug and dosing information; biological pathways (e.g., enriched and dysregulated pathways); and/or adverse data (e.g., data associated with drug safety and tolerability).

### Application Data

Historical clinical trial dataset (e.g., real-world and publication data from other publicly available databases) may be used as a stand in for active trial data.

### Data Harmonization

In some embodiments, the data across the two datasets is aligned, and imputation is performed for missing values, such as missing biomarker levels or other incomplete records.

### Identifying Hidden Patterns and Subgroups Using Unsupervised Learning

In some embodiments, the AI system is configured to cluster patients based on their genomic profiles, other biomarker levels or ALSFRS-R scores or others to uncover new subgroups of patients. Clustering AnswerALS data can identify patient subgroups with distinct molecular profiles (enriched/dysregulated profiles, such as oxidative stress, mitochondrial dysfunction and others). These clusters can be mapped to clinical trial participants using shared features like available biomarkers to retrospectively simulate stratification.

The AI system can test whether stratified trials would have shown improved outcomes for specific pathways or patient profiles. As an example, when considering whether patients in an Edaravone trial, where Edaravone acts as an antioxidant, reducing oxidative stress in neurons, it may not be clear whether the patients in the trial had oxidative stress as a dominant pathway dysregulation. Using clustering from AnswerALS data, the AI system may identify subgroups where oxidative stress pathways are enriched and retrospectively map these subgroups to Edaravone trial participants. The AI system may reveal whether patients with oxidative stress as a dominant mechanism showed better outcomes, and whether stratifying future trials by pathway enrichment could improve drug efficacy. Additionally, for non-responders, the AI system may explore other pathways or dosing strategies that might help identify alternative approaches for improving outcomes.

In live trials, these clusters can be used proactively to stratify participants at enrollment by integrating real time omic data and pathway information, allowing for pathway specific dosing/stratification. Furthermore, in live trials, omics data and other data from participants at enrollment are obtained to identify which pathways are enriched or dysregulated. This data may then be integrated with the existing AnswerALS dataset to create a more robust and comprehensive model. By combining real time data from live trials with the diverse molecular profiles of past ALS patients, the AI system better captures ALS heterogeneity and disease dynamics. This approach will enable the AI system to refine predictions for stratifying participants into more biologically relevant subgroups (also maybe as responders and non-responders to the drug) and tailoring dosing strategies during the trial. Additionally, the AI system may discover novel subgroups that are not defined by traditional responder vs. non-responder classifications but could be useful for refining dosing recommendations or treatment approaches.

### Computer Intelligence Framework

In some embodiments, the AI system performs supervised learning to identify molecular markers specific to ALS patients from AnswerALS omics datasets. It can be similarly applied to other databases and other diseases where molecular profiling is available from both patient and control groups. The AI system is configured to perform data cleansing by imputation of missing values, filtering out less informative molecular markers (absent in majority of samples of one class or the other). The AI system is configured to identify and correct for biases and confounding factors including batch effects across experimental runs, difference in age or gender distribution in patient and control populations etc., using approaches like multivariate regression or naive bayes. The AI system is configured to perform dimensionality reduction to reduce the feature space, to perform classification using Random Forest/logistic regression, and to perform K-fold evaluation and selection of associated features. The AI system is configured to perform high-order machine learning algorithms including boosted trees to check for consistency of associated features between logistic regression/random forest and XGboost routines. The AI system is configured to select significantly associated molecular markers

In some embodiments, the AI system performs unsupervised clustering of patient samples using various combinations of markers identified from multiple omics layers. In some embodiments, the AI system performs K means, UMAP and t-sne based clustering of patients using the selected markers from approach one; data visualization techniques to identify strong associations of various clinical attributes with patient groupings; statistical comparison of molecular markers across groups (ANOVA to compare across groups and t-test for pairwise comparisons) to identify group associated molecular patterns like biological pathways; and/or deep learning to identify patterns of molecular features in each group.

In some embodiments, the AI system is configured to perform feature prioritization based on biological domain understanding and ALS clinical information, such correlations with disease states (ALSFRS), correlation with drug responses, and disease related pathways/tissue expression using Reactome for biological pathways and Protein Atlas for tissue expression inference.

In some embodiments, the AI system is configured to generate a final model to predict patient stratification or drug responses. The AI system includes a multi-class prediction model to predict disease state or patient response to drugs. The AI system may include a supervised classification model that is trained based on data labels from retrospective trials, and the supervised classification model may perform patient classification (drug responders or non-responders) and/or may perform disease state classification based on unseen data or future trials.

For predicting disease states, molecular markers from operation 1 and 2 can be used to build a boosted tree-based ML model to predict the disease state. The model may be evaluated using K-fold method using the same cohort (AnswerALS) and/or an independent cohort (targetALS).

### ALS Example Machine Learning Framework

### Supervised Learning for Identifying Molecular Markers in ALS Patients

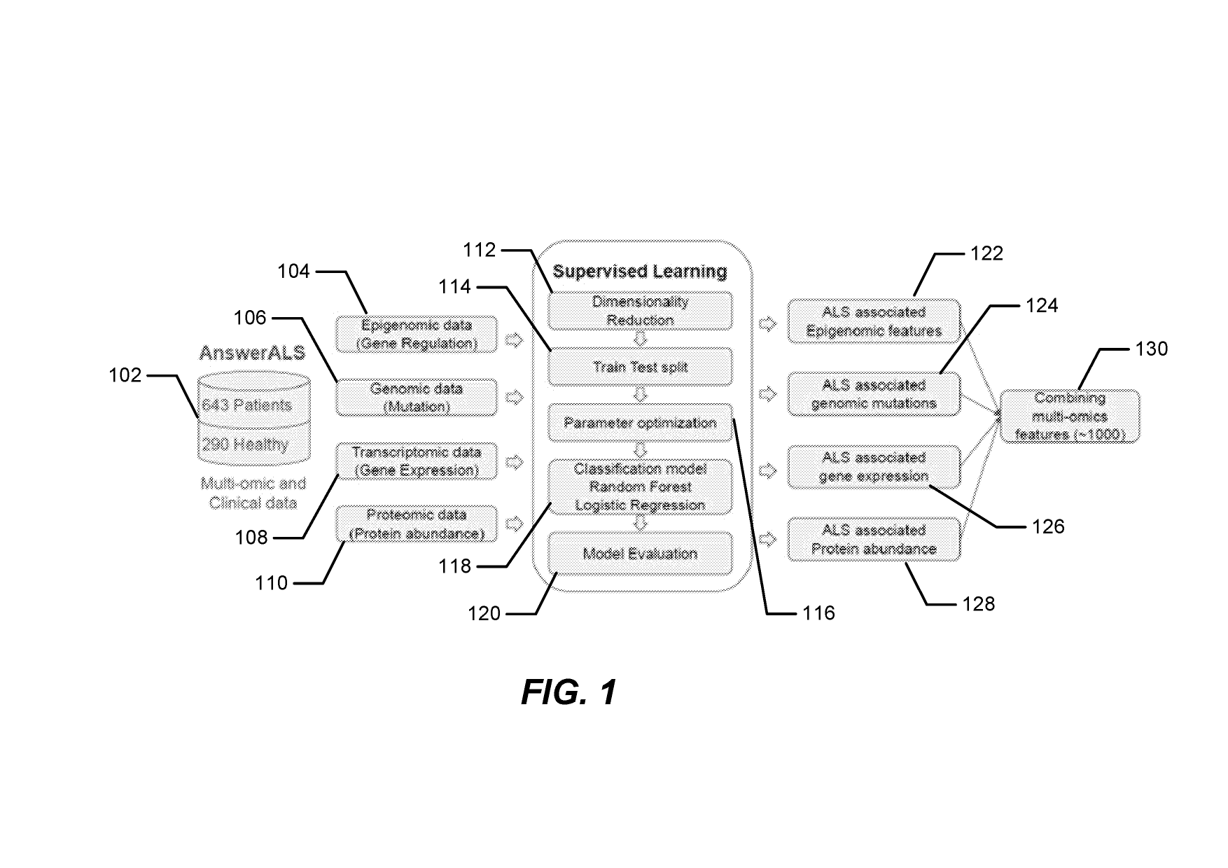

Referring now to FIG. 1, the initial operation in the analysis involves applying supervised machine learning to identify molecular markers that are specific to ALS patients using the AnswerALS multi-omics dataset. While the example embodiments described identifying molecular markers for ALS, the embodiments described herein are applicable to any other diseases where molecular profiling data is available for both patient and control groups.

The AI system is configured to identify ALS-specific molecular signatures that can distinguish affected individuals from healthy controls, ultimately enhancing diagnostic capabilities and informing treatment approaches. The AnswerALS dataset (block 102) comprises 643 patient samples and 290 control samples, totaling over 900 samples. The multi-omics data

includes a variety of molecular layers, such as genomics (block 106), epigenomics (block 104), transcriptomics (block 108), and proteomics (block 110) offering a comprehensive view of ALS at the molecular level.

Before applying machine learning algorithms, the AI system performs a data cleansing routine to ensure the dataset is accurate and consistent. One challenge in working with high-throughput omics data is the presence of missing values, which can arise due to various reasons like sequencing limitations or technical errors. To address this, the AI system applies imputation techniques to estimate missing data based on the most similar samples available. In addition to missing values, some molecular markers may be present in only a small proportion of samples in one class (ALS or control), making them less useful for classification. The AI system may filter out these low-information markers to reduce noise and increase the accuracy of our models.

Given that biological and technical biases can skew results, it may be desirable to address any potential confounding factors. One such factor is the batch effect, which refers to unwanted variability introduced by different experimental conditions or sequencing runs. The AI system is configured to apply ComBat and multivariate regression methods to correct for these biases and ensure that the data is normalized across experimental batches.

Additionally, demographic factors such as differences in age and gender distribution between ALS patients and control groups can confound results, leading to misleading associations. To account for these variations, the AI system uses statistical techniques such as Naïve Bayes classification and multivariate regression to adjust for these demographic differences. This ensures that any identified molecular markers are directly associated with ALS, rather than demographic biases.

Because the multi-omics data is inherently high-dimensional, the AI system may perform dimensionality reduction techniques to simplify the dataset and improve the performance of machine learning models (block 112). Principal Component Analysis (PCA) may be used to identify the primary components of variance in the data, which will help focus on the most relevant features while reducing noise from less informative ones. PCA a dimensionality reduction technique used to simplify large, complex datasets by transforming numerous correlated variables into a smaller set of uncorrelated variables called principal components (PCs). PCA retains maximum variance from the original data. Feature selection may also be performed, which targets markers that show significant differential expression between ALS patients and controls. Correlation-based filtering may be used to remove redundant features, which reduces the risk of overfitting and enhances the efficiency of the AI system.

Thus, according to some embodiments of the disclosure, the features used during the training of the AI systems including machine-learned models disclosed herein may be evaluated and one or more of the features with a lesser relative impact on a prediction, estimation, categorization, inference, or the like than other features may be removed as part of a model sparsification process during both training and inference mode to sparsify the machine-learned model. When the machine-learned model comprises a neural network, one or more nodes or paths may be removed based on the elimination of one or more features. Advantageously, sparsification of the machine-learned model and/or transformer may allow the model to execute using less memory and reduced computational requirements.

For the classification of ALS patients versus controls, the AI system may use multiple machine learning algorithms (blocks 114, 116, 118). Random Forest classifiers may be used due to its robustness in handling high-dimensional, noisy data and its ability to provide insight into which features are desirable for distinguishing ALS from controls. In addition, the AI system may use Logistic Regression (LogReg) as a baseline classifier given its simplicity and interpretability. To evaluate the performance and reliability of the AI system, K-fold cross-validation may be performed to ensure that the classifier generalizes well to unseen data and prevents overfitting.

To further strengthen the analysis, the AI system may perform boosted tree models, such as XGBoost, to refine feature selection by assigning higher importance to the most informative markers across multiple iterations. The feature selection results from Random Forest, Logistic Regression, and XGBoost may be compared to verify consistency in identifying ALS-associated biomarkers. This operation will ensure that the detectable molecular features are robust and reproducible across different methods.

Following feature selection (blocks 122, 124, 126, 128, 130), the AI system may focus on significantly associated molecular markers. The relevance scores generated by Random Forest and XGBoost may help identify the markers with the highest predictive value for ALS. To validate the statistical significance of these markers, ANOVA and t-tests may be conducted to compare the expression levels of these markers between ALS patients and controls (block 120). Additionally, a pathway enrichment analysis to determine if the selected markers are involved in biological pathways previously implicated in ALS, such as neuroinflammation, mitochondrial dysfunction, and RNA processing defects.

### Unsupervised Learning for Identifying Patient Subgroups

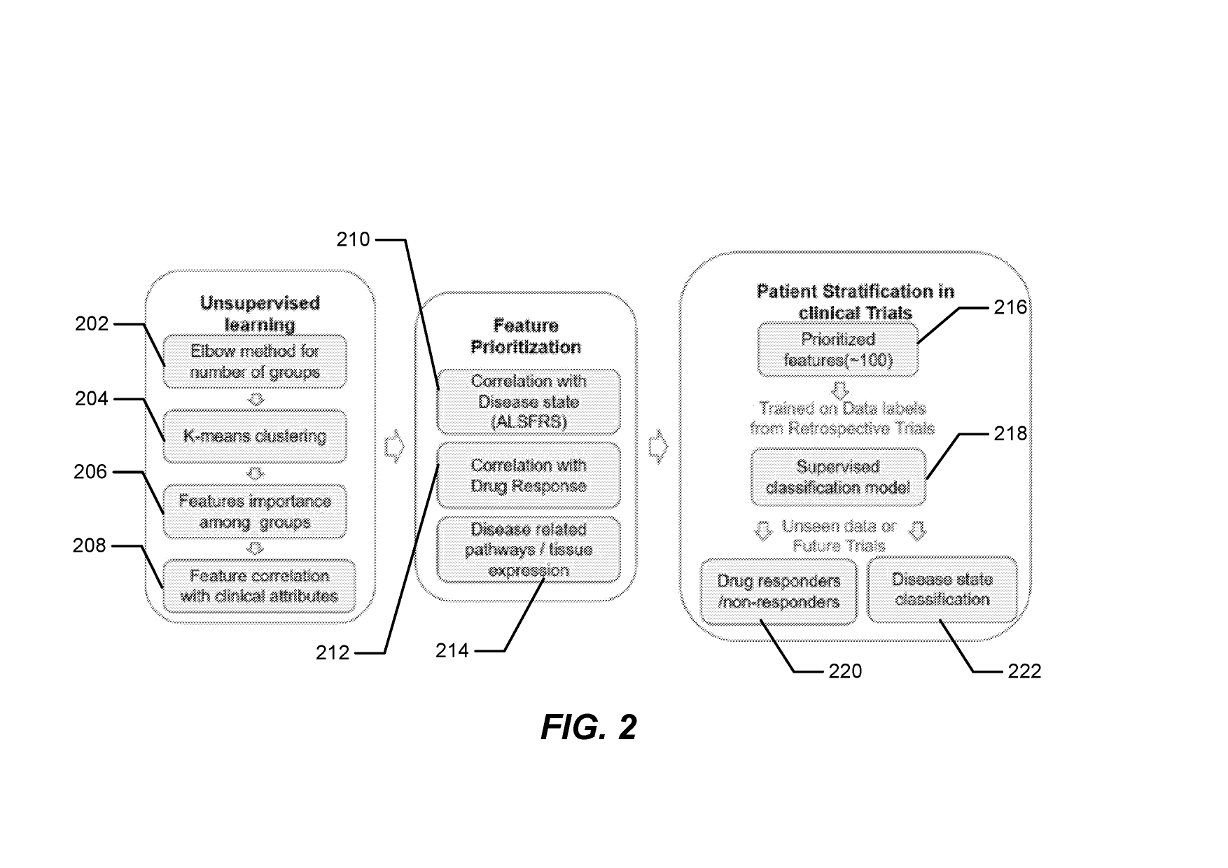

Referring to FIG. 2, once the AI system of FIG. 1 identifies relevant molecular markers, the AI system applies unsupervised clustering techniques to group ALS patients based on their molecular profiles (block 202). Advantageously, by using the first AI system of FIG. 1 to generate a refined feature group of molecular markers for training the second AI system of FIG. 2, the processor time and memory resources used in training the AI system of FIG. 2 can be reduced due to starting with an improved training feature set. Different combinations of biomarkers derived from the multi-omics layers, including genomics, epigenomics, transcriptomics, and proteomics may be explored to uncover distinct patient subgroups with different molecular characteristics. The AI system begins by using K-means clustering and then applying UMAP and t-SNE (t-distributed stochastic neighbor embedding) for dimensionality reduction and visualization of the data (block 204). These methods enable one to visually assess how well the molecular markers separate ALS patients into distinct subgroups and explore the potential patterns within the data.

Following clustering, the AI system may use visualization tools such as heatmaps and scatter plots to assess the relationship between patient groups and clinical attributes, including disease severity, treatment response, and demographic factors (blocks 206, 208). These visualizations will provide insights into how different molecular features correlate with clinical characteristics, further supporting our understanding of disease heterogeneity.

Statistical comparisons may be performed to validate the clusters formed by unsupervised learning. Specifically, ANOVA may be used to identify significant differences between clusters and t-tests for pairwise comparisons to pinpoint the molecular features that drive the formation of these groups. This analysis will help determine whether the clusters align with known biological processes and pathways.

In some embodiments, the AI system may apply deep learning techniques to uncover hidden molecular patterns within each patient group. Using neural networks, the AI system may identify intricate features that define each subgroup and predict patient outcomes. This deep learning approach enhances the detection of meaningful molecular signatures, improves disease classification, and provides a more comprehensive understanding of ALS at the molecular level.

By implementing this rigorous machine learning pipeline, ALS-specific molecular markers may be identified that may serve as diagnostic tools and contribute to early detection, patient stratification, and targeted therapeutic approaches. Furthermore, this methodology is adaptable to other neurodegenerative diseases, making it a valuable tool for biomarker discovery across diverse disorders where molecular profiling data is available. Ultimately, these findings enable one to tailor treatments to individual patients, thereby advancing the field of precision medicine.

The markers identified in this operation will serve as a superset of ALS disease markers and will be a foundation to optimize ongoing and future clinical trials. Given the complexity of ALS, prioritizing the most biologically and clinically relevant molecular markers is desirable. With the vast amount of multi-omics data processed using machine learning, it is desirable to focus on features that have the highest potential for clinical application. The AI system may combine biological domain knowledge with the clinical expertise of ALS specialists, ensuring that the prioritized biomarkers are both biologically meaningful and clinically actionable. This collaborative approach may uncover the most promising biomarkers for ALS monitoring.

### Feature Prioritization through integration of domain knowledge and clinical understanding of the disease

Once the AI system identifies the initial set of ALS-associated features, the AI system may refine and prioritize them based on their biological relevance and clinical applicability. This prioritization process may integrate domain expertise, validation through existing literature, and functional annotations to ensure that the selected markers are both statistically significant and biologically meaningful. The AI system may consider at least the following aspects: correlation with disease state, correlation with drug response, and association with disease-related pathways and tissue expression.

One factor in feature prioritization is evaluating how well a biomarker correlates with ALS disease severity and progression (block 210). The ALS Functional Rating Scale (ALSFRS) is a clinical tool that measures functional decline in ALS patients. The AI system may analyze the correlation between biomarker expression levels and ALSFRS scores to identify markers that consistently associate with different disease progression rates. This analysis will help distinguish between early-stage ALS, slow-progressing ALS, and rapidly progressing ALS. Features that show strong correlations with disease severity may be prioritized as potential prognostic biomarkers, providing valuable insights for disease monitoring and patient stratification.

Another aspect of feature prioritization is understanding how biomarkers correlate with drug response (block 212). Since ALS is a heterogeneous disease, patients exhibit varied responses to treatments. Identifying molecular features that predict therapeutic outcomes is desirable for advancing precision medicine. To achieve this, the AI system may leverage AnswerALS drug response data and information from TargetALS to identify biomarkers linked to either positive or negative treatment effects. Features that demonstrate predictive value for therapy response may be prioritized, allowing for more personalized and targeted treatment strategies.

To further refine the feature selection, the AI system may assess the involvement of identified biomarkers in neuronal pathways and tissue-specific expression, ensuring their direct relevance to ALS pathology (block 214). Using Reactome, the AI system may determine whether selected features are enriched in ALS-relevant biological pathways, such as neuroinflammation, mitochondrial dysfunction, RNA metabolism defects, and synaptic degeneration. These features may be cross-referenced with the Human Protein Atlas to confirm their expression in neuronal tissues, motor neurons, and spinal cord regions—the primary sites affected in ALS. Prioritizing features that align with established ALS mechanisms may enhance their biological and clinical significance, ensuring that the final set of biomarkers is both actionable and impactful in ALS patient care.

### Building a ML model for predicting drug response and disease state

To enhance the efficiency of ALS clinical trials, a supervised classification model may operate and be trained based on prioritized biological features in the previous operation (block 216, 218). These features may be selected based on their relevance to ALS pathology, disease progression, and drug response. The classification model may be trained using labeled data from retrospective study (AnswerALS), where drug response and disease state classifications will either be extracted directly from study datasets or determined by subject matter experts (SMEs) using available clinical information (blocks 220, 222). This classification model may be built using a supervised ML approach (e.g., a multiclass model) and may be built using high order ML algorithms including boosted trees. To evaluate the model beyond the k-fold validation, a subset of patient samples may be excluded from any training operation. This unseen data may indicate the efficacy of the model on any live or future clinical trials. This ML model may be deployed for applicability in other clinical trials.

### Patient Stratification Using Prioritized Features and Disease State Parameters

Patient stratification is a relevant component for advancing precision medicine for ALS. The AnswerALS dataset provides extensive clinical data, including ALSFRS (Amyotrophic Lateral Sclerosis Functional Rating Scale) scores, which are desirable for understanding disease progression. The ALSFRS is widely used to measure the functional abilities of ALS patients, assessing aspects like speech, swallowing, walking, and hand function. By incorporating ALSFRS scores, the AI system can align molecular features with functional impairments to better classify patients into distinct subgroups based on disease state.

Once the biomarkers are prioritized through machine learning, a multiclass classification model may be developed that can predict and distinguish between different ALS subtypes based on the features. This model will allow for accurate predictions of disease state and progression, facilitating more targeted treatment plans for patients and ensures that the classification model is not only data-driven but also clinically relevant, making it a useful tool for precision medicine. This model will offer insights into how ALS progresses, improving our ability to tailor interventions and enhance patient care.

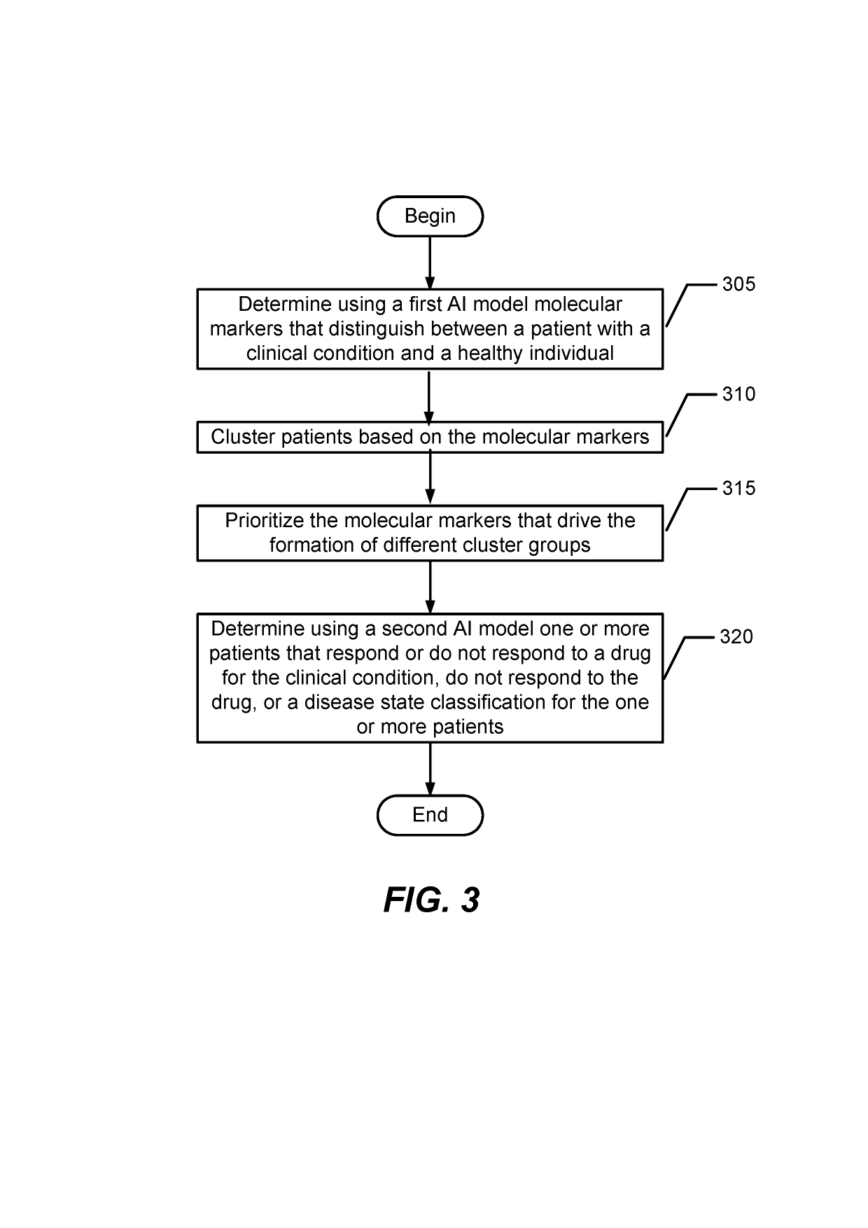

Thus, referring to FIG. 3, operations for molecular marker feature generation, feature prioritization, and/or patient stratification based on drug response and/or disease state classification in a clinical trial begin at block 305 where a first AI model that is trained using multi-omic features associated with a clinical condition is used to determine molecular markers that distinguish between a patient affected with the clinical condition and a healthy individual. Patients are clustered at block 310 based on the molecular markers to determine which of the molecular markers drive the formation of different cluster groups. At block 310 the molecular markers that drive the formation of different cluster groups are prioritized based on biological relevance and clinical applicability;. A second AI model that is trained based on historical prioritized molecular markers and labeled drug response and disease state classifications is used to determine one or more patients that respond to a drug for the clinical condition, one or more patients that do not respond to the drug for the clinical condition, or a disease state classification for each of one or more patients at block 320.

### Potential application in optimization of live clinical trials, patient stratification, and/or doping optimization

In future clinical trials, such as those designed with platform trials, basket trials, adaptive trials, longitudinal studies, observational studies, among others, ALS patients are expected to be recruited, and biological samples may be collected to establish a comprehensive baseline. Multi-omic data, integrating genomic, proteomic, and clinical biomarkers, may be generated and analyzed using an advanced machine learning model trained on extensive historical ALS datasets. During this pre-treatment phase, the model is anticipated to classify patients based on their likelihood of responding to the drug, enabling intelligent trial arm assignment and optimizing patient stratification before the trial progresses.

As the trial advances into the treatment phase, patients will be monitored at scheduled intervals, where additional biological samples may be collected for reassessment. This approach is expected to enable real-time evaluation of early efficacy signals, offering insights into the drug’s therapeutic impact. At least one clinical indicator, such as ALSFRS scores, will be analyzed to determine disease progression and treatment response. The machine learning model will continuously refine patient stratification, ensuring dynamic and data-driven decision-making. If strong efficacy signals are detected, the trial design could be adapted in real time, enrolling more patients with similar biomarker profiles to validate and expand promising results. Conversely, if a lack of therapeutic benefit is identified, the study could be discontinued ahead of schedule, minimizing patient exposure to ineffective treatments and accelerating the development of alternative therapies. This adaptive and AI-enhanced clinical trial model is expected to significantly improve trial efficiency by eliminating reliance on endpoint-only decision-making.

In some embodiments, the model predicts which patients are likely to be responders or non-responders, thereby allowing for optimized trial arm assignment. Once the drug is administered (post-intervention phase), patient samples are collected again at an interim checkpoint, and multi-omic data is reanalyzed. The goal is to detect early efficacy signals in patients, determining whether the drug is providing a meaningful benefit, better improvement in clinical scores/ALSFRS scores. Based on these findings, patient stratification is refined in real time as opposed to waiting until the end of the trial to take decisions. Non-responders are reassessed to identify enriched or dysregulated pathways that could indicate why they are not responding. If an alternative drug is available in the pipeline that targets these pathways, this insight can help guide the next operations in treatment optimization.

### Expanding ML for Disease Diagnostics, Progression, and Outcomes Prediction

The machine learning (ML) models of the AI system can be expanded beyond early-stage disease detection to provide a comprehensive diagnostic and prognostic framework. By integrating multi-omic data, imaging, clinical history, and real-world evidence, the ML models is configured to predict and/or determine disease onset, progression trajectories, and patient-specific outcomes. This allows for early intervention, risk stratification, and real-time monitoring of disease evolution. Advanced AI techniques, including longitudinal modeling and time-series analysis, enable dynamic updates to predictions as new data becomes available. Explainable AI (XAI) ensures model transparency, supporting clinical decision-making while allowing for integration into digital health ecosystems for continuous patient monitoring and personalized care.

### Selecting the Right Drug Among Existing Medications

The AI system enables precision medicine by selecting the most suitable drug from existing medications based on patient-specific molecular and clinical profiles. By analyzing data such as multi-omic data and past drug-response data, the ML model of the AI system may predict the most effective treatment while minimizing adverse effects. It supports real-time adaptation to emerging treatment data, facilitating personalized drug selection and dose optimization. The integration of Explainable AI (XAI) ensures transparency in drug recommendations, aiding clinicians in making data-driven therapeutic decisions that align with patient-specific disease progression and treatment responses.

Embodiments according to the present disclosure provides a technical improvement in clinical trial optimization by transforming heterogeneous biological, clinical, and operational data into persistent optimization-policy objects. These objects remain valid across retrospective, pre-enrollment, and interim inference cycles, enabling cross-study reuse of explainability-derived insights to configure future clinical trial parameters including enrichment thresholds, dosing strategies, arm allocation, and cohort design.

Unlike conventional systems that produce post-hoc analytic results without continuity, the disclosed embodiments serialize optimization-policy objects so decision frameworks may be inherited between studies. This persistence facilitates informed stratification in future trials with or without modifying active clinical protocols, preserving compatibility with regulatory pathways.

Existing multi-omic platforms identify disease mechanisms but do not convert multi-omic signals into reusable trial-design levers. In contrast, the disclosed system, according to some embodiments, integrates genetic variation, protein impact, biomarker patterns, and clinical trajectories to directly influence prospective optimization of trial success metrics including endpoint probability, responder enrichment ratios, and effect-size projection.

Explainability herein is not limited to model interpretation; justification objects generated by explainable artificial intelligence may constrain or modify optimization-policy objects, ensuring that decisions are mechanistically or statistically grounded. These justification objects serve as regulatory-aligned validation artifacts that support traceability matrices, rationale documents, and structured model-feature linkage.

The disclosed embodiments apply to pre-trial enrichment simulations, post-hoc responder analysis, and interim inference cycles without requiring intervention in an ongoing study. This design enables optimization of trial workflows while remaining compatible with regulatory restrictions on live protocol modification and facilitates transmission of actionable learning between trials.

These embodiments improve computer functionality by enabling structured, persistent storage of decision-policy objects that may be reused and refined across studies, reducing redundant computation and enabling automated synthesis of trial-design artifacts previously constructed manually.

### 1. Individual-Level + Cohort-Level Coverage

In addition to the individual-level analyses described in the attached Rancho document, embodiments of the disclosure cover both individual-subject–level and cohort/group-level data analysis. The system may operate on subject-level features, aggregated cohort features, or any hybrid combination. A ‘cohort’ refers to any group of subjects, patients, samples, trial arms, clinical sites, or populations sharing biological, clinical, molecular, demographic, or computational characteristics.

### 2. Predictive + Exploratory + Hybrid Embodiments

Embodiments of the disclosure support predictive, exploratory, and hybrid analytical modes. Predictive embodiments include progression forecasting, risk estimation, outcome trajectory modeling, and cluster-based prediction. Exploratory embodiments include unsupervised clustering, subtype discovery, biomarker associations, dimensionality reduction, network extraction, and heterogeneity mapping. All such modes fall within the intended scope of the embodiments disclosed herein.

### 3. Research-Use and Non-SaMD Embodiments

In certain embodiments, outputs are population-level, exploratory, statistical, or descriptive in nature. These embodiments do not generate individual-level diagnostic, treatment, or patient-management recommendations. Such configurations are intended to remain outside the definition of Software as a Medical Device (SaMD).

### 4. All Clinical Trial Timing Scenarios Pre-Trial + Live-Trial + Post-Trial

The system may operate on data obtained at any stage of a clinical or research workflow;
(a) Pre-trial data: historical datasets, baseline measurements, feasibility data, simulations, or synthetic cohorts.
(b) Live trial data: interim analyses, repeated measurements, rolling enrollment data, adaptive-design updates, real-time or near-real-time streams.
(c) Post-trial data: completed study datasets, long-term follow-up, observational cohorts, or retrospective analyses.

### All timing modes are included within the scope.

### 5. Broad Data Modalities Any Source, Any Origin

Embodiments of the disclosure cover any dataset from clinical trials, observational studies, registries, EHR systems, real-world evidence, biobanks, wearables, mobile devices, digital platforms, sensors, environmental sources, imaging systems, or synthetic/simulated generators.

### 6. Raw Inputs + Engineered Features + Learned Representation

The system may consume raw, minimally processed, engineered, or representation-learned features at any granularity, including token-level, pixel-level, sequence-level, waveform-level, numerical, graphical, or tensor-based formats.

### 7. Broad Modeling Approaches Algorithm-Agnostic

Modeling approaches include supervised, unsupervised, semi-supervised, contrastive, generative, probabilistic, self-supervised, reinforcement-learning, or hybrid models. Embodiments disclosed herein are not limited to HYDRA, GWAS, pQTL, decision trees, XGBoost, neural networks, or any named architecture. End-to-end models, transformers, recurrent models, GNNs, autoencoders, diffusion models, or any computational method are included.

### 8. Objective-Function-Agnostic Design

The modeling engine may optimize any objective function, including prediction-based, metric-learning, contrastive-learning, representation-learning, clustering, or generative objectives.

### 9. Knowledge Graphs + Graph Neural Networks + Network Models

Embodiments may include graph-based analytics such as knowledge graphs, graph neural networks, diffusion models, network embeddings, pathway graphs, and relational or topological data structures.

### 10. Federated, Distributed, Privacy-Preserving & Encrypted Analytics

The system may operate in centralized, federated, decentralized, encrypted, privacy-preserving, homomorphically encrypted, or partially distributed computing environments.

### 11. Multi-Outcome, Multi-Task, Multi-Label Support

The system may support single-task, multi-task, multi-outcome, multi-label, or hierarchical prediction or analysis.

### 12. Continual, Online, Real-Time, or Incremental Learning

Models may be static, periodically updated, or dynamically updated using online, incremental, continual, or real-time learning techniques.

### 13. Output-Agnostic Coverage All Output Types

Outputs may include predictions, scores, cluster assignments, trajectories, embeddings, similarity matrices, visualizations, pathway graphs, network structures, latent variables, or any descriptive or computational representation.

### 14. Disease-Agnostic Framework

Although ALS is used as an example, embodiments of the disclosure apply to any disease or phenotype with measurable biological, clinical, molecular, or digital features.

### 15. Deployment Neutrality

The system may be deployed via cloud infrastructure, APIs, microservices, containerized systems, distributed engines, embedded systems, mobile applications, or edge devices.

### 16. Figure Applicability Across Embodiments

Any diagrams included may represent individual, cohort, predictive, exploratory, online-learning, or hybrid configurations.

The disclosed system, according to some embodiments, applies to any therapeutic domain, including but not limited to rare diseases, oncology, infectious diseases, autoimmune disorders, cardiovascular diseases, neurodegenerative diseases, and metabolic disorders. The computational architecture supports trial design, enrichment, stratification, and optimization independent of specific therapeutic modality, mechanism of action, biomarker class, or biological substrate.

Embodiments of the disclosure introduce optimization artifacts and serializable decision-policy objects that may be preserved post-hoc and subsequently applied to future prospective or active trials, enabling adaptive decision making without requiring real-time access to clinical operations. The system includes mechanisms to ensure that computational inference outputs are represented as structured, machine-readable objects that constrain or inform future cohort selection, dosing strategies, arm allocation, or trial stopping decisions, representing a technical improvement over prior art that only outputs human-readable insights.

The framework supports multi-omic fusion, including single-cell omics, imaging omics, real-world evidence signals, wearable device biometrics, and any future data modalities capable of informing patient stratification and optimization routines. Embodiments of the disclosure therefore protect computational transformations that produce translatable trial decision artifacts, distinct from mere analysis, and enables inference pipelines suitable for storage, retrieval, versioning, and reuse across multiple therapeutic programs and trial cycles.

### Pre-Trial and Post-Hoc Embodiments

Embodiments of the disclosure encompass computational systems and methods that generate reusable decision artifacts for both pre-trial configuration and post-hoc analysis of clinical data. These embodiments ensure that trial optimization is not constrained by temporal stage, and protects workflows irrespective of whether data is historical, interim, or prospective.

Pre-trial embodiments include, but are not limited to: patient stratification based on multi-omic, clinical, and non-clinical data streams; cohort enrichment using computational inference models; simulation or recommendation of dosing schemas prior to subject enrollment; and generation of structured optimization policies that influence trial arm allocation, stopping rules, or recruitment targets. The system produces machine-readable artifacts representing stratification or optimization policies that may be applied to guide future trial design.

Post-hoc embodiments include analysis of completed or partially completed trials to extract reusable decision policies from aggregated datasets, including multi-omic, clinical, imaging, digital biomarker, and real-world data. Computational pipelines extract optimization policies, response signatures, and cohort-specific enrichment signals that may be serialized for reuse in future therapeutic programs. These embodiments generate inference objects capable of informing trial configuration decisions independent of live-trial intervention.

The combination of pre-trial and post-hoc embodiments enables a unified computational architecture that protects optimization of clinical trial design, analysis, and interpretation across disease areas. The system therefore prevents circumvention by entities performing similar computational transformations at alternate temporal stages.

In all embodiments, computational inference pipelines are designed to provide technical improvements over prior art including, but not limited to, generation of structured decision objects, modular decision policies, and serializable optimization artifacts that may be used to influence clinical trial configurations without requiring human interpretation. These artifacts support future adaptive use cases while enabling post-hoc analysis today.

In certain embodiments, optimization objects are represented in one or more computational modalities including, but not limited to: multidimensional numerical arrays, probabilistic tensors, Bayesian prior vectors, graph embeddings, relational logic structures, decision trees, symbolic rule sets, multi-layer latent vectors, or reinforcement-learning policy objects. In some embodiments, internal representations may evolve between trials while maintaining semantic equivalence, thereby enabling extensibility without functional loss.

In some embodiments, the system maintains persistent state containers that store optimization objects across three learning modes—retrospective/post-hoc learning, pre-enrollment predictive learning, and interim/pseudo-adaptive learning—such that each mode inherits and refines optimization objects derived from earlier modes. This inheritance ensures continuity between post-hoc analysis and future trial enrichment without requiring live intervention in ongoing studies.

In certain embodiments, optimization objects inform clinical trial protocol parameters including arm allocation ratios, enrichment thresholds, inclusion and exclusion criteria, dose banding, endpoint selection and hierarchy balancing, statistical powering assumptions, cohort-level sequencing, and subject recruitment pacing. These protocol-level embodiments apply optimization to trial design structures in addition to biomarker stratification.

In some embodiments, investigators or subject matter experts provide manual adjustments to optimization objects, including threshold manipulation, enrichment weighting, prioritization of mechanistic biomarkers, or override of system-proposed stratification. In certain embodiments, such human adjustments are stored as secondary policy embeddings coupled to primary embeddings, enabling hybrid human–machine decision workflows without diminishing automated optimization capability.

In some embodiments, justification objects derived from explainability modules are transformed into regulatory-aligned validation artifacts including structured rationale documents, traceability matrices linking optimization objects to biological mechanisms, model-feature justification records, and statistical-analysis-plan templates incorporating feature importance. These structured artifacts support regulatory review and enable knowledge transfer across trials.

In certain embodiments, optimization objects may be generated using any one or more data modalities including proteomics, genomics, metabolomics, transcriptomics, imaging, digital biomarkers, clinical outcomes, or longitudinal patient trajectories. In some embodiments, multi-modal integration enhances stratification performance but is not required, enabling embodiments wherein a single modality is sufficient for optimization.

In some embodiments, optimization encompasses logistical parameters including sample-collection schedules, biospecimen routing, site resource allocation, patient retention strategies, assay sequencing, and interim visit timing. These embodiments facilitate operational trial improvements in addition to biological signal-based enrichment.

In certain embodiments, optimization objects incorporate causal-inference models and counterfactual simulations to estimate prospective treatment-response trajectories under alternative stratification strategies, enrichment thresholds, or dose allocations. These embodiments enable optimization beyond predictive inference through evaluation of hypothetical trial outcomes.

In some embodiments, optimization success is evaluated using explicit metrics including predicted probability of meeting the primary endpoint, projected responder-enrichment ratio, treatment-effect size estimation, survival-curve shift prediction, or hazard-ratio improvement projections. In some embodiments, such metrics quantitatively define optimization objectives and support measurement of stratification success.

In some embodiments, interim evaluation of optimization objects generates informational futility insights or early-effect indicators for prospective trial-planning purposes without modifying execution of an active trial. In certain embodiments, such informational signals are preserved as decision-policy objects for use in future trial design.

Persistable decision-policy objects constitute serializable artifacts separable from model implementations and may be stored, reused, or refined across studies. In some embodiments, these policy objects support future continuation claims relating to adaptive inference, cohort rebalancing, mechanistic biomarker refinement, causal graph evolution, and protocol decision synthesis.

In some embodiments, the system provides a technical improvement over prior computational approaches by transforming heterogeneous biological and clinical measurements into structured decision-policy objects, persisting these objects across trial phases to enable cross-study reuse and improved statistical sensitivity, and programmatically generating protocol-design recommendations that reduce manual trial-design burden and computational redundancy. These embodiments improve computer functionality by providing persistent optimization representations that reduce recomputation and enable automated synthesis of trial-design artifacts previously constructed manually.

In some embodiments, optimization objects directly target quantifiable clinical trial success metrics including predicted probability of meeting the primary endpoint, probability-adjusted effect-size projection, and responder-enrichment percentage. These embodiments ensure optimization pertains to prospective clinical trial success measures rather than solely biological classification.

The present disclosure provides a computer-implemented framework configured to generate, persist, and reuse optimization objects that directly correspond to quantifiable clinical trial success metrics. The system thereby transforms heterogeneous biological and operational measurements into structured decision-policy objects that programmatically inform trial design parameters including arm allocation, enrichment thresholds, dosing windows, and stratification categories. These technical improvements reduce computational redundancy by maintaining persistent optimization representations across retrospective, pre-enrollment, and interim analysis states.

### Integrated Pre-Trial, Live-Trial, and Post-Hoc Embodiments

The system described herein encompasses computational modes applied before a clinical trial (pre-enrollment optimization), during an ongoing trial without altering execution (interim/pseudo-adaptive optimization), and after trial completion (retrospective/post-hoc analysis). Optimization objects persist across these modes enabling trial-design adjustments and cohort-stratification logic to refine future studies. Persistent state-containers maintain continuity of decision-policy objects, allowing cross-study enrichment without requiring live intervention in ongoing studies.

### Explainability and Regulatory Artifacts

Explainability outputs are transformed into justification objects that generate regulatory-aligned validation artifacts. These include structured rationale documents, traceability matrices linking optimization objects to biological mechanisms, and model-feature justification records. This embodiment enables explainability to satisfy regulatory transparency while supporting claim enablement and enforceable technical improvement.

### Multi-omic and Single-Modality Support

Although multi-omic integration improves feature discovery and stratification precision, optimization objects may be derived from any single modality including genomics, proteomics, metabolomics, or imaging. This embodiment ensures the system remains functional and novel even in single-modality configurations.

### Protocol-Level Optimization Embodiment

Optimization objects provide parameterizations for trial protocol elements such as dose banding, arm allocation ratios, endpoint hierarchy, visit scheduling, cohort rebalancing, and statistical powering assumptions. This embodiment applies optimization to trial protocol structures beyond biological inference.

### Persistent Optimization Objects

Persistent decision-policy objects are serializable artifacts that can be reused across multiple trials and disease areas. These objects are separable from specific model implementations and may be revised, extended, or contextualized by specialists, enabling continuation claims around adaptive inference, causal refinement, and cohort enrichment logic.

### Deployment- and Modality-Neutral Technical Embodiments

Embodiments of the present disclosure apply to computational systems and methods for transforming heterogeneous biological, clinical, digital, operational, and environmental data into structured, persistent optimization objects that inform clinical trial design and post-trial stratification workflows. The embodiments herein extend applicability across all disease types, computational models, deployment architectures, and trial timing modes.

### Broad Modality Coverage

Data sources include but are not limited to: genomics, proteomics, transcriptomics, metabolomics, epigenomics, lipidomics, spatial omics, single-cell measurements, imaging modalities (MRI, PET, CT, ultrasound), digital biomarkers, wearable-derived sensor streams, environmental exposures, social determinants, EHR data, registry datasets, clinical outcomes, mobile sensor signals, and real-world evidence derived from longitudinal observational cohorts.

### Model-Agnostic Computational Coverage

Computational approaches include supervised, unsupervised, semi-supervised, self-supervised, contrastive, probabilistic graphical models, reinforcement learning, neural networks, graph neural networks, transformers, diffusion models, causal models, continual learning frameworks, and hybrid architectures. Embodiments of the disclosure do not rely on a specific named approach.

### Temporal Scope: Pre-Trial, Live, and Post-Trial

Embodiments cover optimization applied before trial initiation, during interim or pseudo-adaptive assessments with or without modifying protocol execution, and post-hoc after-trial completion. Optimization objects persist across timing modes and remain reusable independent of when initial inference occurred, enabling strategic enrichment in future trial cycles without requiring active-trial modification.

### Serializable Decision Objects (Artifact-Level Protection)

Optimization outputs are transformed into serializable, machine-readable decision-policy artifacts that persist beyond singular analyses. These objects inform future trial design decisions including, but not limited to: enrichment thresholds, arm allocation balancing, dosing strategies, eligibility criteria adjustments, endpoint prioritization, and recruitment pacing. Persistence ensures that learned insights remain usable irrespective of trial phase or computational backend changes.

### Model Decoupling and Extendibility

Optimization objects maintain semantic continuity even if underlying models change. This inhibits or prevents invalidation of decision objects when computational architectures evolve, supporting longevity of the invention across future machine learning paradigms.

### Multi-Domain Trial Optimization

Embodiments according to the disclosure apply optimization to biomarker stratification, cohort balancing, protocol parameterization, site operational planning, and assay scheduling. This comprehensive scope protects optimization extending beyond purely biological interpretation.

### Simulation, Counterfactuals, and Hypothetical Trajectories

Optimization objects may incorporate causal inference models and counterfactual simulations to evaluate hypothetical trajectories of treatment-response outcomes, enabling proactive trial design transformations not restricted to observed data.

### Regulatory-Aware Outputs

Explainability outputs are transformed into justification objects suitable for regulatory review including validation records, model-feature linkage matrices, traceability tables, and reasoning templates. This embodiment provides structured transparency and technical improvement beyond conventional black-box predictive systems.

### Cross-Disease, Cross-Trial, and Cross-Mechanism Portability

Although ALS is used as a working example, embodiments apply to any disease, mechanism of action, or therapeutic modality including small molecules, biologics, cell therapies, gene therapies, digital therapeutics, devices, behavioral interventions, or multimodal combinations.

### System-Level Technical Improvements

The disclosed embodiments improve computer functionality through persistent reuse of structured optimization-state representations, which reduce redundant computation, support automated synthesis of protocol design artifacts, provide structured representation formats enabling cross-domain interoperability, enable optimization under variable data incompleteness, and preserve optimization-state continuity independent of underlying computational architectures.

Thus, embodiments according to the disclosure may provide an intelligent platform for clinical trial management that generates computer readable medium storable objects as a persistent, reusable asset rather than a one-off analysis. The platform transforms heterogeneous biological, clinical, and operational data into optimization-policy objects—structured, serializable decision artifacts that encode cohort definitions, enrichment thresholds, dosing windows, arm allocations, and endpoint strategies. These policy objects are then constrained and validated by justification objects derived from explainable AI, so every decision parameter is tied back to explicit biological mechanisms and statistical evidence, creating built-in regulatory rationale. Critically, both the optimization-policy objects and their justification objects are persisted and versioned across retrospective, pre-enrollment, and interim analysis cycles, enabling cross-trial reuse: insights from one study directly configure the protocol parameters of future trials with or without requiring modification of ongoing studies. This persistent, cross-trial decision layer differentiates the platform, according to some embodiments, from conventional ML analytics that simply output reports or scores.

The disclosed embodiments provide an architecture such that the same computational structures, optimization-policy objects, and justification objects can transition into real-time clinical trial decision support without redesign. The embodiments disclosed herein are therefore not limited to retrospective data mining: by persisting protocol-relevant optimization artifacts and their explanatory rationales, the system is inherently capable of continuous or interim-phase adaptation, including but not limited to patient stratification, arm allocation, and dose window selection during ongoing trials when regulatory and operational conditions permit. In this way, the present disclosure protects the core mechanistic framework and data representations that enable live trial optimization ensuring that later evolution into real-time or dose-optimization modes remains within the scope of the disclosed embodiments.

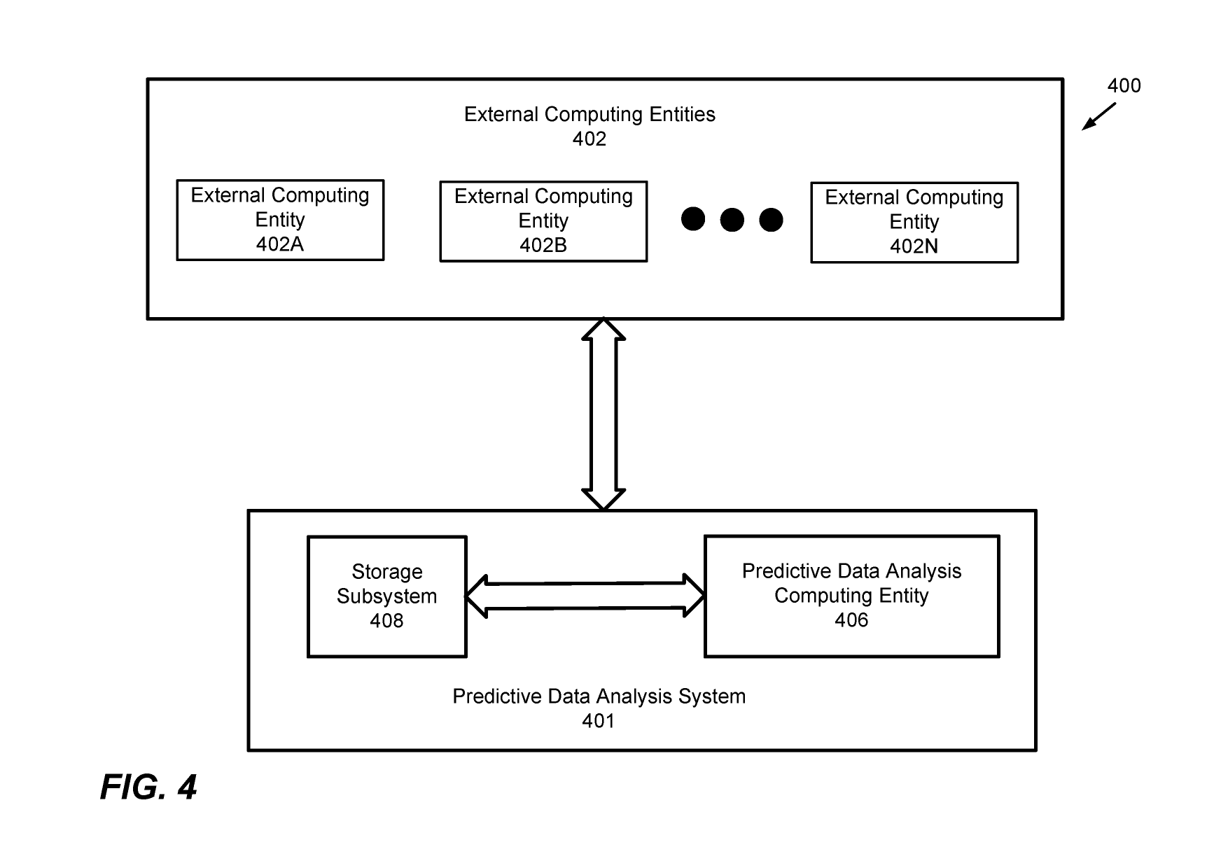

FIG. 4 provides an example overview of a system 1100 that can be used to practice embodiments of the present disclosure. The system 1100 includes a predictive data analysis system 401 comprising a predictive data analysis computing entity 406 configured to generate outputs that can be used to perform one or more output-based actions. The system 1100 may be used to implement one or more of the functional blocks described above with respect to FIGS. 1 – 3. The predictive data analysis system 401 may communicate with one or more external computing entities 402A-N using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (e.g., network routers, and/or the like).

The system 1100 includes a storage subsystem 408 configured to store at least a portion of the data utilized by the predictive data analysis system 401. The predictive data analysis computing entity 406 may be in communication with the external computing entities 402A-N. The predictive data analysis computing entity 406 may be configured to: (i) train one or more machine learning models based on a training data store stored in the storage subsystem 408, (ii) store trained machine learning models as part of a model definition data store of the storage subsystem 408, (iii) use trained machine learning models to perform an action, and/or the like.

In one example, the predictive data analysis computing entity 406 may be configured to generate a prediction, classification, and/or any other data insight based on data provided by an external computing entity such as external computing entity 402A, external computing entity 402B, and/or the like.

The storage subsystem 408 may be configured to store the model definition data store and the training data store for one or more machine learning models. The predictive data analysis computing entity 406 may be configured to receive requests and/or data from at least one of the external computing entities 402A-N, process the requests and/or data to generate outputs (e.g., predictive outputs, classification outputs, and/or the like), and provide the outputs to at least one of the external computing entities 402A-N. In some embodiments, the external computing entity 402A, for example, may periodically update/provide raw and/or processed input data to the predictive data analysis system 401. The external computing entities 402A-N may further generate user interface data (e.g., one or more data objects) corresponding to the outputs and may provide (e.g., transmit, send, and/or the like) the user interface data corresponding with the outputs for presentation to the external computing entity 402A (e.g., to an end-user).

The storage subsystem 408 may be configured to store at least a portion of the data utilized by the predictive data analysis computing entity 406 to perform one or more operations/operations and/or tasks described herein. The storage subsystem 408 may be configured to store at least a portion of operational data and/or operational configuration data including operational instructions and parameters utilized by the predictive data analysis computing entity 406 to perform the one or more operations/operations described herein. The storage subsystem 408 may include one or more storage units, such as multiple distributed storage units that are connected through a computer network. Each storage unit in the storage subsystem 408 may store at least one of one or more data assets and/or one or more data about the computed properties of one or more data assets. Moreover, each storage unit in the storage subsystem 108 may include one or more non-volatile storage or memory media including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

The predictive data analysis computing entity 406 can include an analysis engine and/or a training engine. The predictive analysis engine may be configured to perform one or more data analysis techniques. The training engine may be configured to train the predictive analysis engine in accordance with the training data store stored in the storage subsystem 108.

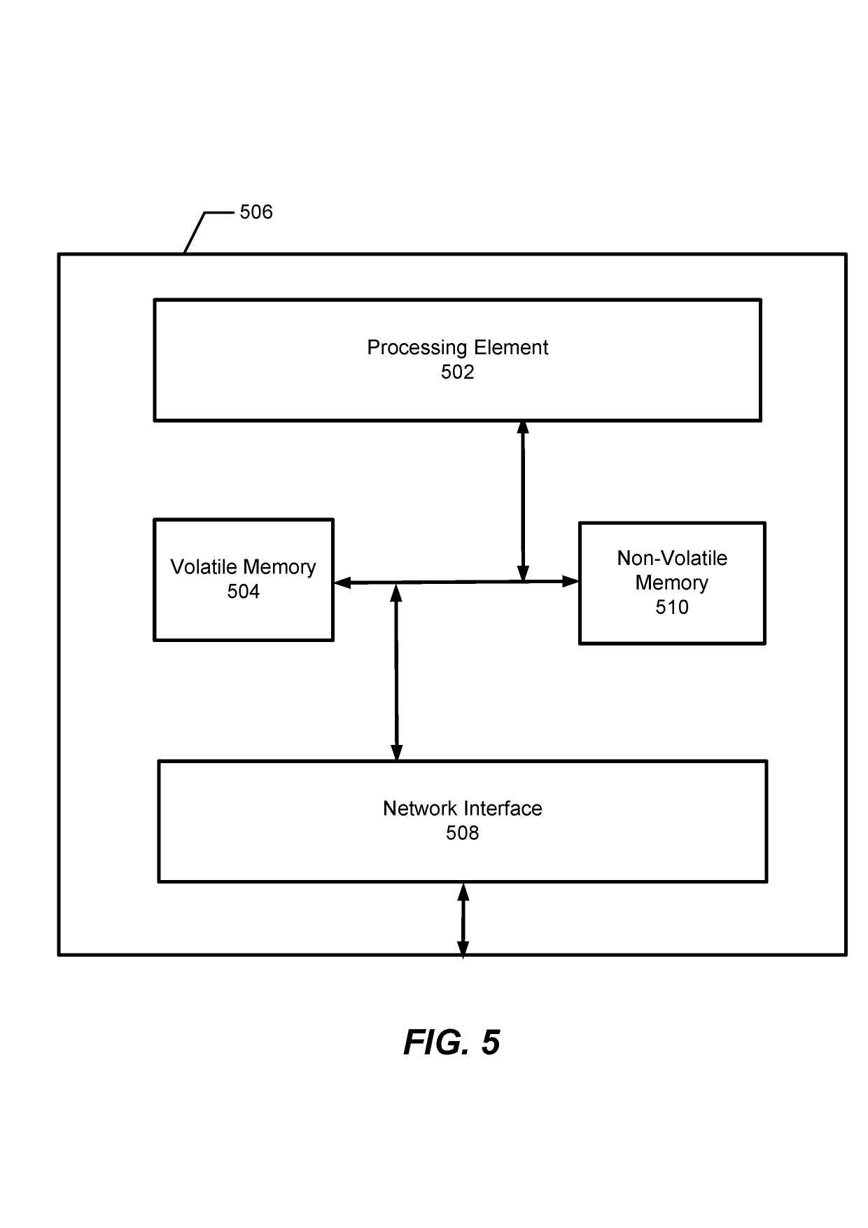

FIG. 5 provides an example predictive data analysis computing entity 506 in accordance with some embodiments discussed herein. In general, the terms computing entity, computer, entity, device, system, and/or similar words used herein interchangeably may refer to, for example, one or more computers, computing entities, desktops, mobile phones, tablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations/operations, and/or processes described herein. Such functions, operations/operations, and/or processes may include, for example, transmitting, receiving, operating on, processing, displaying, storing, determining, creating/generating, monitoring, evaluating, comparing, and/or similar terms used herein interchangeably. In one embodiment, these functions, operations/operations, and/or processes can be performed on data, content, information, and/or similar terms used herein interchangeably.

The predictive data analysis computing entity 506 may include a network interface 508 for communicating with various computing entities, such as by communicating data, content, information, and/or similar terms used herein interchangeably that can be transmitted, received, operated on, processed, displayed, stored, and/or the like.

In one embodiment, the predictive data analysis computing entity 506 may include or be in communication with a processing element 502 (also referred to as processors, processing circuitry, and/or similar terms used herein interchangeably) that communicate with other elements within the predictive data analysis computing entity 506 via a bus, for example. As will be understood, the processing element 502 may be embodied in a number of different ways including, for example, as at least one processor/processing apparatus, one or more processors/processing apparatuses, and/or the like.

For example, the processing element 202 may be embodied as one or more complex programmable logic devices (CPLDs), microprocessors, multi-core processors, coprocessing entities, application-specific instruction-set processors (ASIPs), microcontrollers, and/or controllers. Further, the processing element 502 may be embodied as one or more other processing devices or circuitry. The term circuitry may refer to an entirely hardware embodiment or a combination of hardware and computer program products. Thus, the processing element 202 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 502 may be configured for a particular use or configured to execute instructions stored in one or more memory elements including, for example, one or more volatile memories 504 and/or non-volatile memories 510. As such, whether configured by hardware or computer program products, or by a combination thereof, the processing element 502 may be capable of performing operations or operations according to embodiments of the present disclosure when configured accordingly. The processing element 502, for example in combination with the one or more volatile memories 504 and/or or non-volatile memories 510, may be capable of implementing one or more computer-implemented methods described herein. In some implementations, the predictive data analysis computing entity 506 can include a computing apparatus, the processing element 502 can include at least one processor of the computing apparatus, and the one or more volatile memories 504 and/or non-volatile memories 510 can include at least one memory including program code. The at least one memory and the program code can be configured to, upon execution by the at least one processor, cause the computing apparatus to perform one or more operations/operations described herein.

The non-volatile memories 504 (also referred to as non-volatile storage, memory, memory storage, memory circuitry, media, and/or similar terms used herein interchangeably) may include at least one non-volatile memorydevice204, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile memories 510 may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity–relationship model, object model, document model, semantic model, graph model, and/or the like.

The one or more volatile memories (also referred to as volatile storage, memory, memory storage, memory circuitry, media, and/or similar terms used herein interchangeably) can include at least one volatile memory 504 device, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile memories 504 may be used to store at least portions of the databases, database instances, database management systems, data, applications, programs, program modules, scripts, source code, object code, byte code, compiled code, interpreted code, machine code, executable instructions, and/or the like being executed by, for example, the processing element 502. Thus, the databases, database instances, database management systems, data, applications, programs, program modules, scripts, source code, object code, byte code, compiled code, interpreted code, machine code, executable instructions, and/or the like may be used to control certain embodiments of the operation of the predictive data analysis computing entity 106 with the assistance of the processing element 502.

As indicated, in one embodiment, the predictive data analysis computing entity 506 may also include the network interface 508 for communicating with various computing entities, such as by communicating data, content, information, and/or the like that can be transmitted, received, operated on, processed, displayed, stored, and/or the like. Such communication data may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the predictive data analysis computing entity 106 may be configured to communicate via wireless client communication networks using any of a variety of protocols, such as general packet radio service (GPRS), Universal Mobile Telecommunications System (UMTS), Code Division Multiple Access 2000 (CDMA2000), CDMA2000 1X (1xRTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

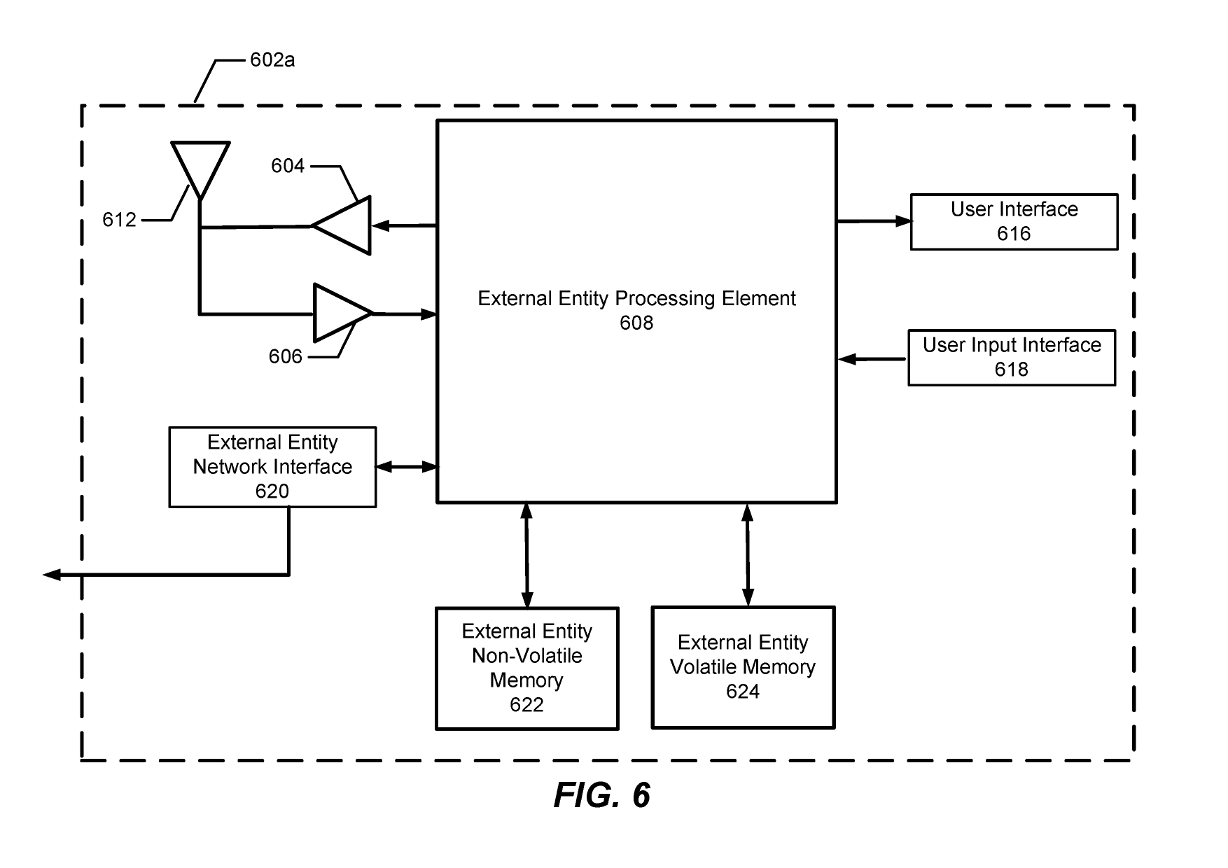

FIG. 6 provides an example external computing entity 602A in accordance with some embodiments discussed herein. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations/operations, and/or processes described herein. The external computing entities 602A-N (corresponding to external computing entities 402A-N can be operated by various parties. As shown in FIG. 6, the external computing entity 602A can include an antenna 612, a transmitter 604 (e.g., radio), a receiver 606 (e.g., radio), and/or an external entity processing element 608 (e.g., CPLDs, microprocessors, multi-core processors, coprocessing entities, ASIPs, microcontrollers, and/or controllers) that provides signals to and receives signals from the transmitter 604 and the receiver 606, correspondingly. As will be understood, the external entity processing element 608 may be embodied in a number of different ways including, for example, as at least one processor/processing apparatus, one or more processors/processing apparatuses, and/or the like as described herein with reference the processing element 502.

The signals provided to and received from the transmitter 604 and the receiver 606, correspondingly, may include signaling information/data in accordance with air interface standards of applicable wireless systems. In this regard, the external computing entity 602A may be capable of operating with one or more air interface standards, communication protocols, modulation types, and access types. More particularly, the external computing entity 602A may operate in accordance with any of a number of wireless communication standards and protocols, such as those described above with regard to the predictive data analysis computing entity 406. In a particular embodiment, the external computing entity 602A may operate in accordance with multiple wireless communication standards and protocols, such as UMTS, CDMA2000, 1xRTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the external computing entity 602A may operate in accordance with multiple wired communication standards and protocols, such as those described above with regard to the predictive data analysis computing entity 406 via an external entity network interface 1320.

Via these communication standards and protocols, the external computing entity 602A can communicate with various other entities using means such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The external computing entity 102A can also download changes, add-ons, and updates, for instance, to its firmware, software (e.g., including executable instructions, applications, program modules), operating system, and/or the like.

According to some embodiments of the disclosure, the external computing entity 602A may include location determining embodiments, devices, modules, functionalities, and/or the like. For example, the external computing entity 602A may include outdoor positioning embodiments, such as a location module adapted to acquire, for example, latitude, longitude, altitude, geocode, course, direction, heading, speed, universal time (UTC), date, and/or various other information/data. In one embodiment, the location module can acquire data such as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating a position of the external computing entity 602A in connection with a variety of other systems, including cellular towers, Wi-Fi access points, and/or the like. Similarly, the external computing entity 602A may include indoor positioning embodiments, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning embodiments can be used in a variety of settings to determine the location of someone or something to within inches or centimeters.

The external computing entity 602A may include a user interface 616 (e.g., a display, speaker, and/or the like) that can be coupled to the external entity processing element 608. In addition, or alternatively, the external computing entity 602A can include a user input interface 618 (e.g., keypad, touch screen, microphone, and/or the like) coupled to the external entity processing element 608).

For example, the user interface 616 may be a user application, browser, and/or similar words used herein interchangeably executing on and/or accessible via the external computing entity 602A to interact with and/or cause the display, announcement, and/or the like of information/data to a user. The user input interface 618 can comprise any of a number of input devices or interfaces allowing the external computing entity 602A to receive data including, as examples, a keypad (hard or soft), a touch display, voice/speech interfaces, motion interfaces, and/or any other input device. In embodiments including a keypad, the keypad can include (or cause display of) the conventional numeric (0-9) and related keys (#, *, and/or the like), and other keys used for operating the external computing entity 602A and may include a full set of alphabetic keys or set of keys that may be activated to provide a full set of alphanumeric keys. In addition to providing input, the user input interface 618 can be used, for example, to activate or deactivate certain functions, such as screen savers, sleep modes, and/or the like.

The external computing entity 602A can also include one or more external entity non-volatile memories 622 and/or one or more external entity volatile memories 624, which can be embedded within and/or may be removable from the external computing entity 602A. As will be understood, the external entity non-volatile memories 622 and/or the external entity volatile memories 624 may be embodied in a number of different ways including, for example, as described herein with reference the volatile memory 504 and/or the non-volatile memory 510.

Some embodiments of the disclosure may provide methods, systems, and computer program products for molecular marker feature generation, feature prioritization, and/or patient stratification based on drug response and/or disease state classification in a clinical trial as set forth in the following examples: Example 1: a computer-implemented method comprises performing, by one or more processors, operations comprising: determining, using a first Artificial Intelligence (AI) model that is trained using multi-omic features associated with a clinical condition, molecular markers that distinguish between a patient affected with the clinical condition and a healthy individual; clustering patients based on the molecular markers to determine which of the molecular markers drive the formation of different cluster groups; prioritizing the molecular markers that drive the formation of different cluster groups based on biological relevance and clinical applicability; and determining, using a second AI model that is trained based on historical prioritized molecular markers and labeled drug response and disease state classifications, one or more patients that respond to a drug for the clinical condition, one or more patients that do not respond to the drug for the clinical condition, or a disease state classification for each of one or more patients.

Example 2: the computer-implemented method of Example 1, wherein the multi-omic features comprise: epigenomic data, genomic data, transcriptomic data, or proteomic data.

Example 3: the computer-implemented method of any of Examples 1 – 2, wherein the method further comprises: processing the multi-omic features to impute estimates of missing data and to remove low-information features.

Example 4: the computer-implemented method of any of Examples 1 – 3, wherein the method further comprises: processing the multi-omic features to remove demographic effects.

Example 5: the computer-implemented method of any of Examples 1 – 4, wherein the method further comprises: reducing the dimensionality of the multi-omic features by removing a subset of the multi-omic features based on a Principal Component Analysis (PCA).

Example 6: the computer-implemented method of any of Examples 1 – 5, wherein the first AI model is configured to place greater importance on ones of the molecular markers that are more informative than other ones of the molecular markers.

Example 7: the computer-implemented method of any of Examples 1 – 6, wherein determining the molecular markers that drive the formation of the different cluster groups comprises: determining the relative of importance of the molecular markers among different cluster groups; and determining a correlation between the molecular markers and clinical attributes.

Example 8: the computer-implemented method of any of Examples 1 – 7, wherein prioritizing the molecular markers comprises: correlating the molecular markers with the severity and progression of the clinical condition; corelating the molecular markers with response to drug treatment; and assessing the involvement of the molecular markers in neuronal pathways and tissue-specific expression.

Example 9: the computer-implemented method of any of Examples 1 – 8, wherein the method further comprises: storing one or more persistent storage objects in a computer readable medium, the persistent storage objects comprising: a patient stratification object based on a multi-omic data stream, a clinical data stream, or a non-clinical data stream; a cohort enrichment object based on one or more computational inference models; a dosing schema object; a policy object directed to clinical trial arm allocation, stopping rules, or recruitment targets; or a post clinical trial analysis object comprising reusable decision policies and optimization policies.

Example 10: the computer-implemented method of any of Examples 1 – 9, wherein the method further comprises: storing one or more persistent optimization objects in a computer readable medium, the optimization objects comprising: retrospective learning objects; pre-enrollment learning objects; and interim learning objects.

Example 11: the computer-implemented method of any of Example 10, wherein the persistent optimization objects are supplemented based on user input.

Example 12: the computer-implemented method of any of Examples 10 – 11, wherein the persistent optimization objects further comprise justification objects that link optimization objects to biological mechanisms and feature important templates.

Example 13: the computer-implemented method of any of Examples 10 – 12, wherein the persistent optimization objects are generated using one or more data modalities comprising proteomics, genomics, metabolomics, transcriptomics, or longitudinal patient trajectories.

Example 14: the computer-implemented method of any of Examples 10 – 13, wherein the persistent optimization objects further comprise: causal inference models and counterfactual simulations objects based on alternative stratification strategies, enrichment thresholds, and drug dose allocations.

Example 15: a system comprises one or more processors and one or more memories configured to store processor-executable instructions that, when executed by the one or more processors, cause the one or more processors to perform operations comprising: determining, using a first Artificial Intelligence (AI) model that is trained using multi-omic features associated with a clinical condition, molecular markers that distinguish between a patient affected with the clinical condition and a healthy individual; clustering patients based on the molecular markers to determine which of the molecular markers drive the formation of different cluster groups; prioritizing the molecular markers that drive the formation of different cluster groups based on biological relevance and clinical applicability; and determining, using a second AI model that is trained based on historical prioritized molecular markers and labeled drug response and disease state classifications, one or more patients that respond to a drug for the clinical condition, one or more patients that do not respond to the drug for the clinical condition, or a disease state classification for each of one or more patients.

Example 16: the system of Example 15, wherein the multi-omic features comprise: epigenomic data, genomic data, transcriptomic data, or proteomic data.

Example 17, the system of any of Examples 15 – 16, wherein determining the molecular markers that drive the formation of the different cluster groups comprises: determining the relative of importance of the molecular markers among different cluster groups; and determining a correlation between the molecular markers and clinical attributes.

Example 18: the system of any of Examples 15 – 17, wherein prioritizing the molecular markers comprises: correlating the molecular markers with the severity and progression of the clinical condition; corelating the molecular markers with response to drug treatment; and assessing the involvement of the molecular markers in neuronal pathways and tissue-specific expression.

Example 19: the system of any of Examples 15 – 18, wherein the operations further comprise storing one or more persistent storage objects in a computer readable medium, the persistent storage objects comprising: a patient stratification object based on a multi-omic data stream, a clinical data stream, or a non-clinical data stream; a cohort enrichment object based on one or more computational inference models; a dosing schema object; a policy object directed to clinical trial arm allocation, stopping rules, or recruitment targets; or a post clinical trial analysis object comprising reusable decision policies and optimization policies.

Example 20: the system of any of Examples 15 – 19, wherein the operations further comprise storing one or more persistent optimization objects in a computer readable medium, the optimization objects comprising: retrospective learning objects; pre-enrollment learning objects; and interim learning objects.

Throughout this specification, components, operations, or structures described as a single instance may be implemented as multiple instances. Although individual operations of one or more methods (or processes, techniques, routines, etc.) are illustrated and described as separate operations, two or more of the individual operations may be performed concurrently or otherwise in parallel, and nothing requires that the operations be performed in the order illustrated. Structures and functionality (e.g., operations, operations, blocks) presented as separate components in example configurations may be implemented as a combined structure, functionality, or component. Similarly, structures and functionality presented as a single component may be implemented as separate components. These and other variations, modifications, additions, and improvements fall within the scope of the subject matter herein.

Certain embodiments are described herein as including logic or a number of routines, subroutines, applications, operations, blocks, or instructions. These may constitute and/or be implemented by software (e.g., code embodied on a non-transitory, machine-readable medium), hardware, or a combination thereof. In hardware, the routines, etc., may represent tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware component that operates to perform certain operations as described herein.

In various embodiments, a hardware component may be implemented mechanically or electronically. For example, a hardware component may comprise dedicated circuitry or logic that is permanently configured (e.g., as a special-purpose processor, such as a field programmable gate array (FPGA) or an application-specific integrated circuit (ASIC)) to perform certain operations. A hardware component may also or instead comprise programmable logic or circuitry (e.g., as encompassed within one or more general-purpose processors and/or other programmable processor(s)) that is temporarily configured by software to perform certain operations.

Accordingly, the term "hardware component" should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware components are temporarily configured (e.g., programmed), each of the hardware components need not be configured or instantiated at any one instance in time. For example, where the hardware components include a general-purpose processor configured using software, the general-purpose processor may be configured as respective different hardware components at different times. Software may accordingly configure a processor, for example, to constitute a particular hardware component at one instance of time and to constitute a different hardware component at a different instance of time.

Hardware components can provide information to, and receive information from, other hardware components. Accordingly, the described hardware components may be regarded as being communicatively coupled. Where multiple of such hardware components exist contemporaneously, communications may be achieved through signal transmission (e.g., over appropriate circuits and buses) that connect the hardware components. In embodiments in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information).

As noted above, the various operations of example methods (or processes, techniques, routines, etc.) described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions. The components referred to herein may, in some example embodiments, comprise processor-implemented components.

Moreover, each operation of processes illustrated as logical flow graphs may represent a sequence of operations that can be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described operations can be combined in any order and/or in parallel to implement the processes.

The terms "coupled" and "connected," along with their derivatives, may be used. In particular embodiments, "connected" may be used to indicate that two or more elements are in direct physical or electrical contact with each other, although the context in the description may dictate otherwise when it is apparent that two or more elements are not in direct physical or electrical contact. "Coupled" may mean that two or more elements are in direct physical or electrical contact. However, "coupled" may also mean that two or more elements are not in direct contact with each other, yet still co-operate, transmit between, or interact with each other.

An algorithm may be considered to be a self-consistent sequence of acts or operations leading to a desired result. These include physical manipulations of physical quantities. Usually, though not necessarily, these quantities take the form of electrical, magnetic, or optical signals capable of being stored, transferred, combined, compared, and otherwise manipulated. These signals are commonly referred to as bits, values, elements, symbols, characters, terms, numbers, flags, or the like. It should be understood, however, that all of these and similar terms are to be associated with the appropriate physical quantities and are merely convenient labels applied to these quantities.

Unless specifically stated otherwise, discussions herein using words such as "processing," "computing," "calculating," "determining," "presenting," "displaying," or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to "some embodiments," "one embodiment," "an embodiment," "in some examples," or variations thereof means that a particular element, feature, structure, characteristic, operation, or the like described in connection with the embodiment is included in at least one embodiment, but not every embodiment necessarily includes the particular element, feature, structure, characteristic, operation, or the like. Different instances of such a reference in various places in the specification do not necessarily all refer to the same embodiment, although they may in some cases. Moreover, different instances of such a reference may describe elements, features, structures, characteristics, operations, or the like be combined in any manner as an embodiment.

As used herein, the terms "comprises," "comprising," "includes," "including," "has," "having" or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless the context of use clearly indicates otherwise, "or" refers to an inclusive or and not to an exclusive or. For example, a condition A or B is satisfied by any one of the following: A is true (or present) and B is false (or not present), A is false (or not present) and B is true (or present), and both A and B are true (or present).

The term "set" is intended to mean a collection of elements and can be a null set (i.e., a set containing zero elements) or may comprise one, two, or more elements. A "subset" is intended to mean a collection of elements that are all elements of a set, but that does not include other elements of the set. A first subset of a set may comprise zero, one, or more elements that are also elements of a second subset of the set. The first subset may be said to be a subset of the second subset if all the elements of the first subset are elements of the second subset, while also being a subset of the set. However, if all the elements of the second subset are also elements of the first subset (in addition to all the elements of the first subset being elements of the second subset), the first subset and the second subset are a single subset/not distinct.

For the purposes of the present disclosure, the term "a" or "an" entity refers to one or more of that entity. As such, the terms "a" or "an", "one or more", and "at least one" can be used interchangeably herein unless explicitly contradicted by the specification using the word "only one" or similar. For example, "a first element" may functionally be interpreted as "a first one or more elements" or a "first at least one element." Unless otherwise apparent from the context of use, reference in the present disclosure to a same set of "one or more processors" (or a same "plurality of processors," etc.) performing multiple operations can encompass implementations in which performance of the operations is divided among the processor(s) in any suitable way. For example, "generating, by one or more processors, X; and generating, by the one or more processors, Y" can encompass: (1) implementations in which a first subset of the processors (e.g., in a first computing device) generates X and an entirely distinct, second subset of the processors (e.g., in a different, second computing device) independently generates Y; (2) implementations in which one or more or all of the processor(s) (e.g., one or multiple processors in the same device, or multiple processors distributed among multiple devices) contribute to the generation of X and/or Y; and (3) other variations. This may similarly be applied to any other component or feature similarly recited (e.g., as "a component", "a feature", "one or more components", "one or more features", "a plurality of components", "a plurality of features"). Moreover, the performance of certain of the operations may be distributed among the one or more components, not only residing within a single machine, but deployed across a number of machines. The set of components may be located in a single geographic location (e.g., within a home environment, an office environment, a cloud environment). In other example embodiments, the set of components may be distributed across two or more geographic locations. Further, "a machine-learned model", equivalent terms (e.g., "machine learning model," "machine-learning model," "machine-learned component", "artificial intelligence", "artificial intelligence component"), or species thereof (e.g., "a large language model", "a neural network") may include a single machine-learned model or multiple machine-learned models, such as a pipeline comprising two or more machine-learned models arranged in series and/or parallel, an agentic framework of machine-learned models, or the like.

An "artificial intelligence" or "artificial intelligence component" may comprise a machine-learned model. A machine-learned model may comprise a hardware and/or software architecture having structural hyperparameters defining the model’s architecture and/or one or more parameters (e.g., coefficient(s), weight(s), biase(s), activation function(s) and/or action function type(s) in examples where the activation function and/or function type is determined as part of training, clustering centroid(s)/medoid(s), partition(s), number of trees, tree depth, split parameters) determined as a result of training the machine-learned model based at least in part on training hyperparameters (e.g., for supervised, semi-supervised, and reinforcement learning models) and/or by iteratively operating the machine-learned model according to the training hyperparameters(e.g., for unsupervised machine-learned models).

In some examples, structural hyperparameter(s) may define component(s) of the model’s architecture and/or their configuration/order, such as, for example, the configuration/order specifying which input(s) are provided to one component and which output(s) of that component are provided as input to other component(s) of the machine-learned model; a number, type, and/or configuration of component(s) per layer; a number of layers of the model; a number and/or type of input nodes in an input layer of the model; a number and/or type of nodes in a layer; a number and/or type of output nodes of an output layer of the model; component dimension (e.g., input size versus output size); a number of trees; a maximum tree depth; node split parameters; minimum number of samples in a leaf node of a tree; and/or the like. The component(s) of the model may comprise one or more activation functions and/or activation function type(s) (e.g., gated linear unit (GLU), such as a rectified linear unit (ReLU), leaky RELU, Gaussian error linear unit (GELU), Swish, hyperbolic tangent), one or more attention mechanism and/or attention mechanism types (e.g., self-attention, cross-attention), nodes and split indications and/or probabilities in a decision tree, and/or various other component(s) (e.g., adding and/or normalization layer, pooling layer, filter). Various combinations of any these components (as defined by the structural hyperparameter(s)) may result in different types of model architectures, such as a transformer-based machine-learned model (e.g., encoder-only model(s), encoder-decoder model(s), decoder-only models, generative pre-trained transformer(s) (GPT(s))), neural network(s), multi-layer perceptron(s), Kolmogorov-Arnold network(s), clustering algorithm(s), support vector machine(s), gradient boosting machine(s), and/or the like. The structural parameters and components a machine-learned model comprises may vary depending on the type of machine-learned model.

Training hyperparameter(s) may be used as part of training or otherwise determining the machine-learned model. In some examples, the training hyperparameter(s), in addition to the training data and/or input data, may affect determining the parameter(s) of the target machine-learned model. Using a different set of training hyperparameters to train two machine-learned models that have the same architecture (i.e., the same structural hyperparameters) and using the same training data may result in the parameters of the first machine-learned model differing from the parameters of the second machine-learned model. Despite having the same architecture and having been trained using the same training data, such machine-learned models may generate different outputs from each other, given the same input data. Accordingly, accuracy, precision, recall, and/or bias may vary between such machine-learned models.

In some examples, training hyperparameter(s) may include a train-test split ratio, activation function and/or activation function type (e.g., in examples like Kolmogorov-Arnold networks (KANs) where the activation function type is determined as part of training from an available set of activation functions and/or limits on the activation function parameters specified by the training hyperparameters), training stage(s) (e.g., using a first set of hyperparameters for a first epoch of training, a second set of hyperparameters for a second epoch of training), a batch size and/or number of batches of data in a training epoch, a number of epochs of training, the loss function used (e.g., L1, L2, Huber, Cauchy, cross entropy), the component(s) of the machine-learned model that are altered using the loss for a particular batch or during a particular epoch of training (e.g., some components may be "frozen," meaning their parameters are not altered based on the loss), learning rate, learning rate optimization algorithm type (e.g., gradient descent, adaptive, stochastic) used to determine an alteration to one or more parameters of one or more components of the machine-learned model to reduce the loss determined by the loss function, learning rate scheduling, and/or the like.

In some examples, the structural hyperparameters and/or the training hyperparameters may be determined by a hyperparameter optimization algorithm or based on user input, such as a software component written by a user or generated by a machine-learned model. The machine-learned model may include any type of model configured, trained, and/or the like to generate a prediction output for a model input. In some examples, any of the logic, component(s), routines, and/or the like discussed herein may be implemented as a machine-learned model.

The machine-learned model may include one or more of any type of machine-learned model including one or more supervised, unsupervised, semi-supervised, and/or reinforcement learning models. Training a machine-learned model may comprise altering one or more parameters of the machine-learned model (e.g., using a loss optimization algorithm) to reduce a loss. Depending on whether the machine-learned model is supervised, semi-supervised, unsupervised, etc. this loss may be determined based at least in part on a difference between an output generated by the model and ground truth data (e.g., a label, an indication of an outcome that resulted from a system using the output), a cost function, a fit of the parameter(s) to a set of data, a fit of an output to a set of data, and/or the like. In some examples, determining an output by a machine-learned model may comprise executing a set of inference operations executed by the machine-learned model according to the target machine-learned model’s parameter(s) and structural hyperparameter(s) and using/operating on a set of input data.

Moreover, any discussion of receiving data associated with an individual that may be protected, confidential, or otherwise sensitive information, is understood to have been preceded by transmitting a notice of use of the data to a computing device, account, or other identifier (collectively, "identifier") associated with the individual, receiving an indication of authorization to use the data from the identifier, and/or providing a mechanism by which a user may cause use of the data to cease or a copy of the data to be provided to the user.

Upon reading this disclosure, those of skill in the art will appreciate still additional alternative structural and functional designs through the principles disclosed herein. Therefore, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The patent claims at the end of this patent application are not intended to be construed under 35 U.S.C. § 112(f) unless traditional means-plus-function language is expressly recited, such as "means for" or "operation for" language being explicitly recited in the claim(s).

As used herein, the term "and/or" includes any and all combinations of one or more of the associated listed items.

The embodiments of the present disclosure have been presented for purposes of illustration and description, but are not intended to be exhaustive or limited to the disclosure in the form disclosed. Many modifications and variations may be apparent to those of ordinary skill in the art without departing from the scope and spirit of the disclosure. The aspects of the disclosure herein were chosen and described to best explain the principles of the disclosure and the practical application thereof, and to enable others of ordinary skill in the art to understand the disclosure with various modifications as are suited to the particular use contemplated.

What is claimed is:

1. A computer-implemented method comprises performing, by one or more processors, operations comprising:
determining, using a first Artificial Intelligence (AI) model that is trained using multi-omic features associated with a clinical condition, molecular markers that distinguish between a patient affected with the clinical condition and a healthy individual;
clustering patients based on the molecular markers to determine which of the molecular markers drive the formation of different cluster groups;
prioritizing the molecular markers that drive the formation of different cluster groups based on biological relevance and clinical applicability; and
determining, using a second AI model that is trained based on historical prioritized molecular markers and labeled drug response and disease state classifications, one or more patients that respond to a drug for the clinical condition, one or more patients that do not respond to the drug for the clinical condition, or a disease state classification for each of one or more patients.

2. The method of claim 1, wherein the multi-omic features comprise:
epigenomic data, genomic data, transcriptomic data, or proteomic data.

3. The method of claim 1, further comprising:
processing the multi-omic features to impute estimates of missing data and to remove low-information features.

4. The method of claim 1, further comprising:
processing the multi-omic features to remove demographic effects.

5. The method of claim 1, further comprising:
reducing the dimensionality of the multi-omic features by removing a subset of the multi-omic features based on a Principal Component Analysis (PCA).

6. The method of claim 1, wherein the first AI model is configured to place greater importance on ones of the molecular markers that are more informative than other ones of the molecular markers.

7. The method of claim 1, wherein determining the molecular markers that drive the formation of the different cluster groups comprises:
determining the relative of importance of the molecular markers among different cluster groups; and
determining a correlation between the molecular markers and clinical attributes.

8. The method of claim 1, wherein prioritizing the molecular markers comprises:
correlating the molecular markers with the severity and progression of the clinical condition;
corelating the molecular markers with response to drug treatment; and
assessing the involvement of the molecular markers in neuronal pathways and tissue-specific expression.

9. The method of claim 1, further comprising storing one or more persistent storage objects in a computer readable medium, the persistent storage objects comprising:
a patient stratification object based on a multi-omic data stream, a clinical data stream, or a non-clinical data stream;
a cohort enrichment object based on one or more computational inference models;
a dosing schema object;
a policy object directed to clinical trial arm allocation, stopping rules, or recruitment targets; or
a post clinical trial analysis object comprising reusable decision policies and optimization policies.

10. The method of claim 1, further comprising storing one or more persistent optimization objects in a computer readable medium, the optimization objects comprising:
retrospective learning objects;
pre-enrollment learning objects; and
interim learning objects.

11. The method of claim 10, wherein the persistent optimization objects are supplemented based on user input.

12. The method of claim 10, wherein the persistent optimization objects further comprise justification objects that link optimization objects to biological mechanisms and feature important templates.

13. The method of claim 10, wherein the persistent optimization objects are generated using one or more data modalities comprising proteomics, genomics, metabolomics, transcriptomics, or longitudinal patient trajectories.

14. The method of claim 10, wherein the persistent optimization objects further comprise:
causal inference models and counterfactual simulations objects based on alternative stratification strategies, enrichment thresholds, and drug dose allocations.

15. A system, comprising:
one or more processors; and
one or more memories configured to store processor-executable instructions that, when executed by the one or more processors, cause the one or more processors to perform operations comprising:
determining, using a first Artificial Intelligence (AI) model that is trained using multi-omic features associated with a clinical condition, molecular markers that distinguish between a patient affected with the clinical condition and a healthy individual;
clustering patients based on the molecular markers to determine which of the molecular markers drive the formation of different cluster groups;
prioritizing the molecular markers that drive the formation of different cluster groups based on biological relevance and clinical applicability; and
determining, using a second AI model that is trained based on historical prioritized molecular markers and labeled drug response and disease state classifications, one or more patients that respond to a drug for the clinical condition, one or more patients that do not respond to the drug for the clinical condition, or a disease state classification for each of one or more patients.

16. The system of claim 15, wherein the multi-omic features comprise:
epigenomic data, genomic data, transcriptomic data, or proteomic data.

17. The system of claim 15, wherein determining the molecular markers that drive the formation of the different cluster groups comprises:
determining the relative of importance of the molecular markers among different cluster groups; and
determining a correlation between the molecular markers and clinical attributes.

18. The system of claim 15, wherein prioritizing the molecular markers comprises:
correlating the molecular markers with the severity and progression of the clinical condition;
corelating the molecular markers with response to drug treatment; and
assessing the involvement of the molecular markers in neuronal pathways and tissue-specific expression.

19. The system of claim 15, wherein the operations further comprise storing one or more persistent storage objects in a computer readable medium, the persistent storage objects comprising:
a patient stratification object based on a multi-omic data stream, a clinical data stream, or a non-clinical data stream;
a cohort enrichment object based on one or more computational inference models;
a dosing schema object;
a policy object directed to clinical trial arm allocation, stopping rules, or recruitment targets; or
a post clinical trial analysis object comprising reusable decision policies and optimization policies.

20. The system of claim 15, wherein the operations further comprise storing one or more persistent optimization objects in a computer readable medium, the optimization objects comprising:
retrospective learning objects;
pre-enrollment learning objects; and
interim learning objects.
